SEQUENCE LISTING (8 sequences)

SEQ ID NO: 1 - DNA, 20 bp, Artificial Sequence
  Description of Artificial Sequence Synthetic primer
gaagaacaagcccgaccaga

SEQ ID NO: 2 - DNA, 20 bp, Artificial Sequence
  Description of Artificial Sequence Synthetic primer
ccacctgggtcacactcttc

SEQ ID NO: 3 - DNA, 20 bp, Artificial Sequence
  Description of Artificial Sequence Synthetic primer
tgggctacactgagcaccag

SEQ ID NO: 4 - DNA, 20 bp, Artificial Sequence
  Description of Artificial Sequence Synthetic primer
gggtgtcgctgttgaagtca

SEQ ID NO: 5 - protein, 9 aa, Artificial Sequence
  Description of Artificial Sequence Synthetic peptide
KAVYNFATM

SEQ ID NO: 6 - protein, 11 aa, Artificial Sequence
  Description of Artificial Sequence Synthetic peptide
SGVENPGGYCL

SEQ ID NO: 7 - protein, 9 aa, Artificial Sequence
  Description of Artificial Sequence Synthetic peptide
FQPQNGQFI

SEQ ID NO: 8 - protein, 20 aa, Artificial Sequence
  Description of Artificial Sequence Synthetic peptide
GLNGPDIYKGVYQFKSVEFD

(19) United States
(12) Patent Application Publication
(10) Pub. No.: US 2019/0290728 A1
(43) Pub. Date: Sep. 26, 2019

(54) METHODS RELATED TO BREAKING T CELL EXHAUSTION

(71) Applicant: CHILDREN'S MEDICAL CENTER CORPORATION, Boston, MA (US)

(72) Inventors: Florian WINAU, Boston, MA (US); Yu HU, Malden, MA (US)

(73) Assignee: CHILDREN'S MEDICAL CENTER CORPORATION, Boston, MA (US)

(21) Appl. No.: 16/342,704

(22) PCT Filed: Oct. 20, 2017
(86) PCT No.: PCT/US2017/057534

§ 371 Date: Apr. 17, 2019

Related U.S. Application Data
(60) Provisional application No. 62/410,910, filed on Oct. 21, 2016.

(51) Int. Cl. A61K 38/17; A61K 45/06

(52) U.S. Cl. CPC A61K 38/1738; A61K 45/06

(57) ABSTRACT

Described herein are methods and compostions relating to breaking or inhibiting T cell exhaustion by increasing TMEM16F levels and/or activity. The methods and compositions can thereby further relate to treatment of viral infections or cancer, treatment of a chronic disease, vaccine administration, administering a CAR-T therapy, or increasing the number of T-bet+ T cells in a subject. In some embodiments, the methods relate to treatment of an autoimmune or inflammatory disease by inhibiting TMEM16F levels and/or activity.

### CROSS-REFERENCE TO RELATED APPLICATIONS

This application claims benefit under 35 U.S.C. § 119(e) of U.S. Provisional Application No. 62/410,910 filed Oct. 21, 2016, the contents of which are incorporated herein by reference in their entirety.

### TECHNICAL FIELD

Described herein are methods for, e.g., breaking T cell exhaustion and/or increasing T-bet+ T cell numbers by increasing the level and/or activity of TMEM16F. Such methods can relate to the treatment of chronic diseases, the increase of an immune response, and/or the treatment of a viral infection or cancer.

### BACKGROUND

T cells are vital for virus clearance and inhibition of tumor growth. In patients with chronic infections or cancer, T cells become exhausted and lose the ability to effectively kill the diseased cells, making it impossible for the patient's body to effectively resolve the disease. Reversal of T cell exhaustion, such as by immune checkpoint blockade, provides a novel and efficient way to treat these patients.

### SUMMARY

As described herein, the inventors have discovered that TMEM16F acts to restrict T cell exhaustion. Accordingly, methods relating to increasing the level and/or activity of TMEM16F can break or ameliorate, prevent, and/or reduce T cell exhaustion and can be used to treat conditions such as chronic infections or cancer.

In one aspect of any of the embodiments, described herein is a method of treating a viral infection or cancer in a subject in need thereof, the method comprising administering an agonist of TMEM16F to the subject. In some embodiments of any of the aspects relating to the administration of an agonist of TMEM16F, T cell exhaustion is inhibited.

In one aspect of any of the embodiments, described herein is method of increasing the number of T-bet+ T cells in a subject, the method comprising administering an agonist of TMEM16F to a subject in need thereof.

In some embodiments of any of the aspects relating to the administration of an agonist of TMEM16F, the subject is in need of an increase of an immune response to a chronic disease. In some embodiments of any of the aspects relating to the administration of an agonist of TMEM16F, the subject is in need of treatment for a viral infection or cancer. In some embodiments of any of the aspects, the viral infection is an HIV or hepatitis B virus (HBV). In some embodiments of any of the aspects, the cancer is a recurrent cancer. In some embodiments of any of the aspects, the cancer is selected from the group consisting of melanoma; NSCLC; renal cell carcinoma; Hodgkin lymphoma; and bladder cancer.

In some embodiments of any of the aspects, the agonist of TMEM16F is administered before another cancer treatment. In some embodiments of any of the aspects, the agonist of TMEM16F is administered concurrently with another cancer treatment. In some embodiments of any of the aspects relating to the administration of an agonist of TMEM16F, the subject is further administered an immune checkpoint therapy. In some embodiments of any of the aspects, the immune checkpoint therapy is selected from the group consisting of an anti-PD-1 therapy; an anti-CTLA4 therapy; an anti-PD-L1 therapy; an anti-TIM-3 therapy; and an anti-LAG-3 therapy. In some embodiments of any of the aspects, the anti-PD-1 therapy is selected from the group consisting of nivolumab; and pembrolizumab. In some embodiments of any of the aspects, the anti-CTLA4 therapy is ipilimumab. In some embodiments of any of the aspects, the anti-PD-L1 therapy is atezolizumab. In some embodiments of any of the aspects, the anti-TIM3 therapy is TSR-022. In some embodiments of any of the aspects, the anti-LAG3 therapy is BMS-986016.

In some embodiments of any of the aspects relating to the administration of an agonist of TMEM16F, the subject is further administered a CAR-T cell.

In one aspect of any of the embodiments, described herein is a method of administering a vaccine to a subject in need thereof, the method comprising administering 1) the vaccine and 2) an agonist of TMEM16F to the subject. In one aspect of any of the embodiments, described herein is a composition comprising 1) a vaccine and 2) an agonist of TMEM16F to the subject. In some embodiments of any of the aspects, the vaccine and the agonist of TMEM16F are administered sequentially. In some embodiments of any of the aspects, the vaccine and the agonist of TMEM16F are administered concurrently. In some embodiments of any of the aspects relating to administration of a vaccine, the subject is a subject with reduced immune function. In some embodiments of any of the aspects, the subject with reduced immune function is a subject with a chronic disease.

In one aspect of any of the embodiments, described herein is a method of administering a CAR-T therapy to a subject in need thereof, the method comprising: contacting a CAR-T cell or T cell ex vivo with an agonist of TMEM16F; and administering the CAR-T cell or a CAR-T cell obtained from the T cell to the subject. In some embodiments of any of the aspects, the T-cell is contacted with the agonist of TMEM16F prior to modifiying the T cell to create a CAR-T cell. In some embodiments of any of the aspects, the CAR-T is contacted with the agonist of TMEM16F after modifying a T cell to create a CAR-T cell. In some embodiments of any of the aspects relating to administration of a CAR-T cell, the subject is not administered an agonist of TMEM16F. In some embodiments of any of the aspects, a detectable level of the agonist of TMEM16F is not present in or on the CAR-T cell when the CAR-T cell is administered to the subject.

In one aspect of any of the embodiments, described herein is a method of increasing T-bet+ T cell activity, proliferation, and/or survival, the method comprising contacting a T cell with an agonist of TMEM16F. In one aspect of any of the embodiments, described herein is a method of inhibiting T cell exhaustion, the method comprising contacting a T cell with an agonist of TMEM16F. In some embodiments of any of the aspects, the T cell is a T cell obtained from a subject or a T cell differentiated from a cell obtained from a subject. In some embodiments of any of the aspects, the T cell is administered to a subject after the contacting step.

In one aspect of any of the embodiments, described herein is a method of measuring the activity of a TMEM16F agonist candidate, the method comprising: contacting a membrane comprising TMEM16F and NBD-phospholipids with dithionite and the agonist candidate; and measuring the fluorescence of the NBD-phospholipids; wherein the greater the decrease in the fluorescence, the greater the activity of the agonist candidate. In some embodiments of any of the aspects, the membrane is a liposome. In some embodiments of any of the aspects, the membrane does not comprise another scramblase.

In one aspect of any of the embodiments, described herein is a method of measuring the activity of a TMEM16F agonist candidate, the method comprising: contacting a cell with the agonist candidate; contacting the cell with annexinV; and measuring the level of annexinV staining on the cell surface; wherein the greater the level of staining, the greater the activity of the agonist candidate. In some embodiments of any of the aspects, the first contacting step further comprises contacting the cell with the calcium ionophore A23187.

In some embodiments of any of the aspects, the agonist of TMEM16F is an SSRI inhibitor.

In one aspect of any of the embodiments, described herein is a method of treating an autoimmune or inflammatory disease in a subject in need thereof, the method comprising administering an inhibitor of TMEM16F to the subject. In one embodiment of any of the aspects relating to administration of an inhibitor of TMEM16F, T cell exhaustion is increased.

In one aspect of any of the embodiments, described herein is a method of decreasing the number of T-bet+ T cells in a subject, the method comprising administering an inhibitor of TMEM16F to a subject in need thereof.

In one embodiment of any of the aspects relating to administration of an inhibitor of TMEM16F, the subject is in need of a decrease of an immune response. In one embodiment of any of the aspects relating to administration of an inhibitor of TMEM16F, the subject is in need of treatment for an autoimmune or inflammatory disease. In one embodiment of any of the aspects the autoimmune or inflammatory disease is selected from the group consisting of: inflammatory bowel disease,; type I diabetes; multiple sclerosis; Systemic lupus erythematosus (SLE); Crohn's disease; autoimmune dilated cardiomyopathy; autoimmune myocarditis; autoimmune enteritis; arthritis; rheumatoid arthritis; collagen-induced arthritis; autoimmune hemolytic anemia; autoimmune hepatitis.

In one aspect of any of the embodiments, described herein is a method of decreasing T-bet+ T cell activity, proliferation, and/or survival, the method comprising contacting a T cell with an inhibitor of TMEM16F. In one aspect of any of the embodiments, described herein is a method of decreasing T cell exhaustion, the method comprising contacting a T cell with an inhibitor of TMEM16F.

In some embodiments of any of the aspects, the inhibitor of TMEM16F is an inhibitory nucleic acid; an aptamer; an antibody reagent; an antibody; or a small molecule.

In one aspect of any of the embodiments, described herein is a method of measuring the activity of a TMEM16F inhibitor candidate, the method comprising: contacting a membrane comprising TMEM16F and NBD-phospholipids with dithionite and the inhibitor candidate; and measuring the fluorescence of the NBD-phospholipids; wherein the greater the increase in the fluorescence, the greater the activity of the inhibitor candidate. In some embodiments of any of the aspects, the membrane is a liposome. In some embodiments of any of the aspects, the membrane does not comprise another scramblase.

In one aspect of any of the embodiments, described herein is a method of measuring the activity of a TMEM16F inhibitor candidate, the method comprising: contacting a cell with the inhibitor candidate; contacting the cell with annexinV; and measuring the level of annexinV staining on the cell surface; wherein the lower the level of staining, the greater the activity of the inhibitor candidate. In some embodiments of any of the aspects, the first contacting step further comprises contacting the cell with the calcium ionophore A23187.

### BRIEF DESCRIPTION OF THE DRAWINGS

FIGS. 1A-1B demonstrate that TMEM16F is the dominant scramblase in T cells. FIG. 1A depicts immunoblot analysis of TMEM16F expression in WT or TMEM16F-deficient (KO) thymocytes. FIG. 1B depicts experiments in which PS exposure was examined by annexin-V staining and flow cytometry on splenocytes from TMEM16F-KO or WT mice in response to calcium ionophore A23187. DMSO was used as control. B220, B cells. Data are representative of at least two experiments.

FIGS. 2A-2D demonstrate that lack of TMEM16F causes increased T cell activation. FIGS. 2A-2C depict flow cytometry analysis of intracellular IFN-γ (FIGS. 2A, 1B), and surface CD25 (FIG. 2C) of CD8 T cells activated by GP33. Quantification of IFN-γ-producing cells is shown in (FIG. 2B). MFI, mean fluorescence intensity. Results are displayed as mean±s.e.m. *P<0.05, **P<0.01; ns, not significant, using Student's t-test. In FIG. 2C, KO is shown in grey, WT in black. FIG. 2D depicts phosphorylation of LAT and ERK induced by GP33 stimulation and detected by immunoblot of splenocytes from KO or WT mice. Data are representative of two to six experiments.

FIGS. 3A-3D demonstrate increased T cell activation in TMEM16F-KO mice during early phase of chronic infection. WT or TMEM16F-KO mice were intravenously infected with 4×10⁶ pfu LCMV clone 13. T cell responses were analyzed on day 6 p.i. (FIGS. 3A, 3B) Frequencies of GP33-tetramer-positive cells among total CD8 T cells in (FIG. 3A), and GP66-tetramer-positive cells among total CD4 T cells in (FIG. 3B) were determined by flow cytometry. In FIG. 3C, proliferation of GP33-tetramer-positive T cells from spleen was measured by BrdU incorporation at indicated time points post infection. FIG. 3D depicts flow cytometry analysis of intracellular IFN-γ production in CD8 T cells in response to GP33, or PMA and Ionomycin (P+I). Results are displayed as mean±s.e.m. of two independent experiments (n=3-5 mice per group). *P<0.05, **P<0.01; ns, not significant, using Student's t-test.

FIGS. 4A-4J demonstrate that TMEM16F deficiency causes severe T cell exhaustion. WT or TMEM16F-KO mice were intravenously infected with 4×10⁶ pfu LCMV clone 13 for 80 days. Expression of PD-1 in GP33-tetramer-positive CD8 T cells (FIGS. 4A, 4B) and coproduction of IFN-γ and TNF-α in CD8 T cells from spleen after restimulation with indicated epitopes (FIGS. 4C, 4D) were analyzed by flow cytometry. Expression of PD-1 in GP66-tetramer-positive CD4 T cells (FIGS. 4E, 4F) and coproduction of IFN-γ and TNF-α in CD4 T cells from spleen after restimulation with GP66 (FIGS. 4G, 4H) were analyzed by flow cytometry. In FIGS. 4I-4J, expression of T-bet and Eomes in GP33-tetramer-positive CD8 T were analyzed by flow cytometry. MFI, mean fluorescence intensity. Results are displayed as mean±s.e.m. of two to five independent experiments (n=3-5 mice per group). Student's t-test was used. *P<0.05, **P<0.01, ****P<0.0001.

FIGS. 5A-5E demonstrate that TMEM16F regulates T cell response in a cell-intrinsic manner. WT or TMEM16F-deficient bone marrow (BM) chimeric mice were intravenously infected with 4×10⁶ pfu LCMV clone 13 and sacrificed at day 150 p.i. Expression of PD-1 (FIG. 5A), T-bet and Eomes (FIG. 5C) in GP33-tetramer-positive CD8 T cells, and cytokine production in CD8 T cells after stimulation with GP33 (FIG. 5B) were analyzed by flow cytometry. For FIGS. 5D-5E, mixed BM chimera were infected in a similar fashion as in FIGS. 5A-5C. Mice were sacrificed at day 150 p.i. Cytokine production in WT or KO CD8 T cells after stimulation with GP33 in (FIG. 5D), and expression of T-bet and Eomes in GP33-tetramer-positive CD8 T cells in (FIG. 5E) were determined by flow cytometry. MFI, mean fluorescence intensity. Results are displayed as mean±s.e.m. of two independent experiments (3-4 mice per group). *P<0.05; ***P<0.001; ****P<0.0001; ns, not significant using Student's t-test.

FIGS. 6A-6D demonstrate that TMEM16F is required for control of chronic viral infection. WT or TMEM16F-KO mice were intravenously infected with 4×10⁶ pfu LCMV clone 13 for 80 days (FIGS. 6A, 6B), or 150 days (FIG. 6C). In FIG. 6A, the absolute number of GP33-tetramer-positive CD8 T cells per 1×10⁶ PBMCs was analyzed by flow cytometry at indicated time points post infection. PBMC, peripheral blood mononuclear cell. Virus loads in serum in (FIG. 6B) at indicated time points and in kidney at day 138 post infection in (FIG. 6C) were determined by focus assay. For FIG. 6D, WT or TMEM16F-deficient bone marrow (BM) chimeric mice were intravenously infected with 4×10⁶ pfu LCMV clone 13 and sacrificed at day 150 p.i. Virus loads in kidney and liver of BM chimeric mice were determined by focus assay. LOD, Limit of Detection. Results are displayed as mean±s.e.m. of two to three independent experiments (n=3-5 mice per group). Student's t-test was used. *P<0.05, **P<0.01, ****P<0.0001.

FIGS. 7A-7E demonstrate that TMEM16F is recruited to the synapse and requires microtubules for transport. Jurkat cells expressing TMEM16F-RFP were cocultured with Raji cells (FIG. 7A) or stimulated on coverslips coated with αCD3 (FIGS. 7B-7E). FIG. 7A depicts confocal microscopy analysis for localization of TMEM16F in Jurkat T cells cocultured with control (unpulsed), or Staphylococcal enterotoxin E (SEE)-pulsed Raji B cells (SEE+). 2 μm Z-stack of images is shown. DIC, differential interference contrast. FIGS. 7B-7C depict the dynamics of TMEM16F at the TCR activation site imaged by TIRF microscopy. Number of TMEM16F-positive spots was quantified in (FIG. 7C). FIGS. 7D-7E depict the eynamics of TMEM16F at the TCR activation site imaged by TIRF microscopy in Jurkat cells pretreated with vehicle (DMSO), or 1 μM nocodazole, or 1 blebbistatin. FIG. 7D depicts the number of TMEM16F-positive spots was quantified. Time zero is the start of recording. FIG. 7E depicts the trajectories of TMEM16F-positive spots tracked by ImageJ. Scale bars, 5 μm. Data are representative of three independent experiments.

FIGS. 8A-8C demonstrate that TMEM16F resides in late but not early or recycling endosomes. Jurkat cells expressing TMEM16F-RFP together with Rab7-GFP in (FIG. 8A), Rab5-GFP in (FIG. 8B), and Rab11-GFP in (FIG. 8C) were stimulated on coverslips coated with αCD3. Cells were imaged by TIRF. Shown is the spatial correlation of localization of TMEM16F and Rab7 (FIG. 8A), Rab5 (FIG. 8B), or Rab11 (FIG. 8C) upon TCR stimulation. The fluorescence intensities (pixels) along the dotted line were determined by ImageJ.

FIGS. 9A-9E demonstrate that TMEM16F is involved in MVB formation upon TCR engagement. FIG. 9B depicts confocal microscopy analysis of LBPA staining of Jurkat cells seeded on coverslips coated with αCD45 (non-stimulatory) or αCD3 (stimulatory). Number of LBPA-positive vesicles was quantified. FIG. 9B depicts quantification of LBPA-positive vesicles in non-target (control) or T16F-KD (TMEM16F-knockdown) Jurkat T cells treated with αCD45 or αCD3. FIGS. 9C-9E depict electron microscopy of MVBs in non-target or T16F-KD Jurkat T cells. Representative electron micrographs are shown in FIG. 9C. Arrows indicate MVBs. Scale bars, 100 nm. Quantification of the number of intraluminal vesicles (ILVs) per MVB and categorization of MVB stages are shown in (FIG. 9D) and (FIG. 9E), respectively. Results are displayed as mean±s.e.m. **P<0.01, ***P<0.001, ****P<0.0001; ns, not significant, using Student's t-test. Data are representative of three experiments.

FIGS. 10A-10F demonstrate that impaired cSMAC formation and sustained TCR signaling in TMEM16F-silenced T cells. FIGS. 10A-10B depict confocal microscopy analysis of cSMAC formation by measuring centralized accumulation of TCR-β at the IS between Jurkat T cells and SEE-pulsed Raji B cells. Percentage of cSMAC-positive cells is shown in (FIG. 10B). Insets are 3D reconstructions of the IS en face. Non, control; KD, TMEM16F-knockdown. DIC, differential interference contrast. FIGS. 10C-10D depict non-target or T16F-KD Jurkat cells expressing mCherry-tSH2-ZAP70 were stimulated on αCD3-coated coverslips. Dynamics of mCherry-tSH2-ZAP70 at the TCR activation site were imaged by TIRF microscopy. SH2 domain of ZAP70 specifically binds to phosphorylated tyrosine residues in the CD3 signaling units of TCRs. Signal from the phosphotyrosine probe is depicted as thermal intensity in FIG. 10C. Number of mCherry-tSH2-ZAP70-positive spots were quantified in FIG. 10D. FIGS. 10E-10F depict dynamics of LAT microclusters at the TCR activation site were imaged by TIRF microscopy. Number of LAT microclusters is quantified in FIG. 10F. Scale bars, 5 μm. Data are representative of three experiments.

FIGS. 11A-11D demonstrate normal T cell development in TMEM16F-KO mice. FIG. 11A depicts total lymphocyte numbers of thymus and spleen from WT or TMEM16F-KO mice. Results are displayed as mean±s.e.m. Depicted are frequencies of conventional T cells in thymus, spleen, and lymph nodes (LNs) in FIG. 11B, Treg cells in spleen in FIG. 11C, and liver invariant NKT cells in FIG. 11D as assessed by flow cytometry. Data are representative of two to four independent experiments (n=4-5 mice per group). Student's t-test was used. ns, not significant.

FIG. 12 depicts a schematic mechanistic model. Upper panel: TCR engagement, via increased intracellular Ca²⁺ levels, activates scramblase TMEM16F in late endosomes to mediate the formation of MVBs. Newly generated MVBs sequester intracellular TCR signaling complexes for subsequent lysosomal degradation to terminate T cell activation. This TMEM16F-mediated checkpoint determines the duration of signaling and the proper ratio of T-bet^{hi} to Eomes^{hi} effector T cells to facilitate virus clearance. Lower panel: In the absence of TMEM16F, generation of MVBs is hampered, TCR signaling molecules accumulate, and T cell activation is sustained. Breaking the TMEM16F checkpoint leads to prolonged signaling that shifts the balance towards terminally differentiated Eomes^{hi} T cells and ultimate loss of virus protection.

FIGS. 13A-13B depict experimental procedures. FIG. 13A depicts exemplary data in which TMEMB16F activity is measured via PS exposure for annexin-V staining FIG. 13B depicts the production of bone marrow chimeric mice expressing TMEM16F-CA, and treatment of the mice to examine the effect of TMEM16F overactivation on development of T cell exhaustion and virus control.

FIG. 14A depicts a model of T cell exhaustion regulation by TMEM16F. FIG. 14B depicts an experimental approach to increase TMEM16F expression during LCMV-C13 infection with PD-1 blockade, thereby modulating T cell exhaustion and virus clearance.

FIG. 15 depicts schematics relating to the PS-annexin-V assays for measuring TMEM16F activity described herein.

### DETAILED DESCRIPTION

As described herein, the inventors have demonstrated that increased levels and/or activity of TMEM16F negatively regulates T cell exhaustion. Thus, agonists of TMEM16F can break or inhibit T cell exhaustion, increase the number and/or proportion of T-bet+ T cells, and/or increase an immune response. Accordingly, described herein are methods relating to increasing TMEM16F levels and/or activity, e.g., in order to treat chronic diseases, viral infections, and/or cancer.

As used herein, “TMEM16F,” “transmembrane protein 16F,” or “anoctamin 6” refers to a calcium-dependent scramblase that can move phosphatidylserine (PS) from an inner membrane surface to an outer membrane surface and/or from an outer membrane surface to an inner membrane surface. Sequences for TMEM16F are known for a number of species, e.g., human TMEM16F (NCBI Gene ID: 196527) mRNA sequences (NM_001025356.2, NM_001142678.1, NM_001142679.1, and NM_001204803.1) and polypeptide sequences (NP_001191732.1, NP_001136151.1, NP_001136150.1, and NP_001020527.2).

As used herein, the term “agonist” refers to an agent which increases the expression and/or activity of the target by at least 10% or more, e.g. by 10% or more, 50% or more, 100% or more, 200% or more, 500% or more, or 1000% or more. The efficacy of an agonist of, for example, TMEM16F, e.g. its ability to increase the level and/or activity of TMEM16F can be determined, e.g. by measuring the level of an expression product of TMEM16F and/or the activity of TMEM16F. Methods for measuring the level of a given mRNA and/or polypeptide are known to one of skill in the art, e.g. RTPCR with primers can be used to determine the level of RNA, and Western blotting with an antibody can be used to determine the level of a polypeptide. Non-limiting examples of suitable primers are provided in the Examples herein and antibodies to TMEM16F are commercially available, e.g., Cat. No. sc-136930 from Santa Cruz Biotechnology (Dallas, Tex.). Assays for measuring the activity of TMEM16F, e.g. the level of PS being moved from one membrane surface to another are provided elsewhere herein.

Non-limiting examples of agonists of a given target, e.g., TMEM16F, can include TMEM16F polypeptides or variants or functional fragments thereof and nucleic acids encoding a TMEM16F polypeptide or variants or functional fragments thereof.

In some embodiments of any of the aspects, the agonist of, e.g. TMEM16F can be a TMEM16F polypeptide. In some embodiments of any of the aspects, the polypeptide agonist can be an engineered and/or recombinant polypeptide. In some embodiments of any of the aspects, the polypeptide agonist can be a nucleic acid encoding a polypeptide, e.g. a functional fragment thereof. In some embodiments of any of the aspects, the nucleic acid can be comprised by a vector.

In some embodiments of any of the aspects, the agonist of TMEM16F can be a TMEM16F polypeptide, e.g., exogenous TMEM16F polypeptide. In some embodiments of any of the aspects, the target cell(s) and/or subject is contacted with and/or administered exogenous TMEM16F polypeptide, e.g., TMEM16F polypeptide is produced in vitro and/or synthesized and purified TMEM16F polypeptide is provided to the target cell(s) and/or subject.

In some embodiments of any of the aspects, a TMEM16F agonist can be a polypeptide comprising the sequence of a human TMEM16F polypeptide, e.g., any of NP_001191732.1, NP_001136151.1, NP_001136150.1, and NP_001020527.2. In some embodiments of any of the aspects, a TMEM16F agonist can be a nucleic acid comprising a sequence which encodes a human TMEM16F polypeptide, e.g., any of NP_001191732.1, NP_001136151.1, NP_001136150.1, and NP_001020527.2.

In some embodiments of any of the aspects, the agonist of TMEM16F can be a nucleic acid encoding a TMEM16F polypeptide, e.g., exogenous and/or ectopic TMEM16F polypeptide. In some embodiments of any of the aspects, the target cell(s) and/or subject is contacted with and/or administered the nucleic acid encoding exogenous and/or ectopic TMEM16F polypeptide, e.g., the nucleic acid is transcribed and/or translated after the contacting or administering step to provide exogenous and/or ectopic TMEM16F to the target cell(s) and/or subject.

In some embodiments of any of the aspects, the agonist of TMEM16F can be a nucleic acid encoding a polypeptide comprising the sequence of TMEM16F (or a variant or functional fragment thereof) and/or a vector comprising a nucleic acid encoding a polypeptide comprising the sequence of TMEM16F (or a variant or functional fragment thereof). A nucleic acid encoding a polypeptide can be, e.g., an RNA molecule, a plasmid, and/or an expression vector. In some embodiments of any of the aspects, the nucleic acid encoding a polypeptide can be an mRNA. In some embodiments of any of the aspects, the nucleic acid encoding a polypeptide can be a modified mRNA.

In some embodiments of any of the aspects, an agonist of TMEM16F can be an SSRI (Selective serotonin re-uptake inhibitor). SSRI's can facilitate TMEM16F ion current activation (see, e.g., Kim et al. Pflugers Arch 2015 467:2243-2256; which is incorporated by reference herein in its entirety). SSRI's are well known in the art and can include, by way of non-limiting example, fluoxetine, sertraline, and paroxetine. Further non-limiting examples of SSRIs can include citalopram, escitalopram, fluvoxamine, dapoxetine, indalpine, zimelidine, cericlamine, and panuramine.

In one aspect of any of the embodiments, described herein is a method of treating a viral infection or cancer in a subject in need thereof, the method comprising administering an agonist of TMEM16F to the subject. In one aspect of any of the embodiments, described herein is a method of treating a chronic disease in a subject in need thereof, the method comprising administering an agonist of TMEM16F to the subject.

As used herein, “chronic disease” refers to a disease that persists for an extended period of time. In some embodiments of any of the aspects, the chronic disease can be an infection or cancer.

Chronic diseases can include chronic or persistent infections, e.g., those infections that, in contrast to acute infections, are not effectively cleared by the induction of a naturally occurring host immune response. During such persistent infections, the infectious agent and the immune response reach equilibrium such that the infected subject remains infected over a long period of time without necessarily expressing symptoms. Persistent infections can involve stages of both silent and productive infection without rapidly killing or even producing excessive damage of the host cells. Persistent infections include for example, latent, chronic and slow infections. In some embodiments of any of the aspects, the infection can be infection with a bacterium, virus, fungus, or parasite.

In a “chronic infection,” the infectious agent is present in the subject at all times. However, the signs and symptoms of the disease can be present or absent for an extended period of time. Non-limiting examples of chronic infection include hepatitis B (caused by hepatitis B virus (HBV)) and hepatitis C (caused by hepatitis C virus (HCV)), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus II. Parasitic persistent infections can arise as a result of infection by, for example, Leishmania, Toxoplasma, Trypanosoma, Plasmodium, Schistosoma, and Encephalitozoon. In some embodiments of any of the aspects, a chronic infection can be an infection with viruses including, but not limited to, human T-Cell leukemia viruses, Epstein-Barr virus, cytomegalovirus, herpesviruses, varicella-zoster virus, measles, papovaviruses, prions, hepatitis viruses, HIV, adenoviruses, parvoviruses and papillomaviruses.

In some embodiments of any of the aspects, a chronic infection can be a latent infection. In some embodiments of any of the aspects, a chronic infection can include periods in which the infection is a latent infection. In a “latent infection,” the infectious agent (such as a virus) is seemingly inactive and dormant such that the subject does not always exhibit signs or symptoms. In a latent viral infection, the virus remains in equilibrium with the host for long periods of time before symptoms again appear; however, the actual viruses cannot typically be detected until reactivation of the disease occurs. Non-limiting examples of latent infections include infections caused by herpes simplex virus (HSV)-1 (fever blisters), HSV-2 (genital herpes), and varicella zoster virus VZV (chickenpox-shingles).

In a “slow infection,” the infectious agents gradually increase in number over a very long period of time during which no significant signs or symptoms are observed. Non-limiting examples of slow infections include AIDS (caused by HIV-1 and HIV-2), lentiviruses that cause tumors in animals, and prions.

Examples of infectious viruses include, but are not limited to: Retroviridae (for example, HIV); Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections, with these and other viral agents.

Examples of fungal infections include but are not limited to: aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. The compositions and methods described herein are contemplated for use in treating infections with these and other fungal agents.

Examples of infectious bacteria include, but are not limited to: Helicobacterpyloris, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces Israelli. The compositions and methods described herein are contemplated for use in treating infections with these and other bacterial agents.

Other infectious organisms (such as protists) include: Plasmodium falciparum and Toxoplasma gondii. The compositions and methods described herein are contemplated for use in treating infections with these and other agents.

In some embodiments of the aspects described herein, the methods further comprise administering an effective amount of a viral, bacterial, fungal, or parasitic treatment in conjunction with an agonist of TMEM16F.

As used herein, the term “cancer” relates generally to a class of diseases or conditions in which abnormal cells divide without control and can invade nearby tissues. Cancer cells can also spread to other parts of the body through the blood and lymph systems. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably. A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice. See, e.g., Freshney, CULTURE ANIMAL CELLS: MANUAL BASIC TECH. (3rd ed., 1994).

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

In some embodiments of any of the aspects, the cancer can be melanoma, NSCLC, renal cell carcinoma, Hodgkin lymphoma, or bladder cancer. Further examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments of any of the aspects, the agonist of TMEM16F can be administered to increase the immune response, e.g., concurrently or sequentially with another cancer therapy, e.g., a cancer therapy not targeting the immune response. In some embodiments of any of the aspects, the agonist of TMEM16F can be administered before a second cancer therapy. In some embodiments of any of the aspects, the agonist of TMEM16F can be administered concurrently with a second cancer therapy. In some embodiments of any of the aspects, the cancer can be a recurrent cancer.

In one aspect of any of the embodiments, described herein is a method of increasing an immune response in a subject in need thereof, the method comprising administering an agonist of TMEM16F to the subject. In one aspect of any of the embodiments, described herein is a method of increasing an immune response in a subject in need of treatment for a chronic disease, viral infection, and/or cancer, the method comprising administering an agonist of TMEM16F to the subject. An increase in an immune response can include the level and/or activity of any aspect of the immune system, e.g., a cell, a cytokine, or activity thereof

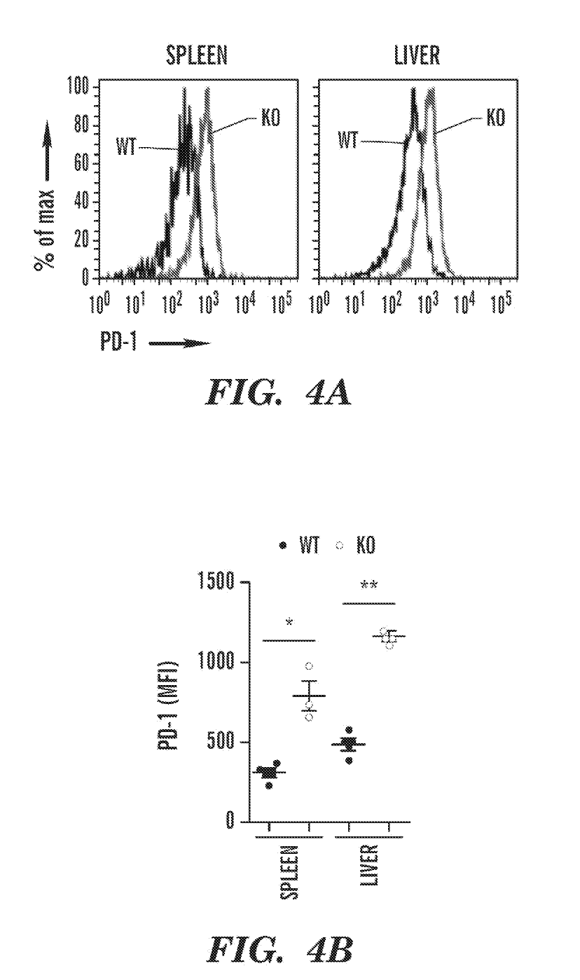

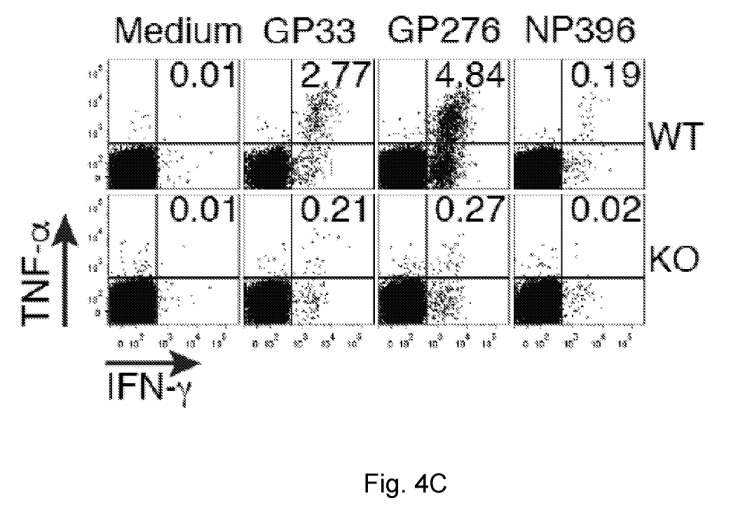

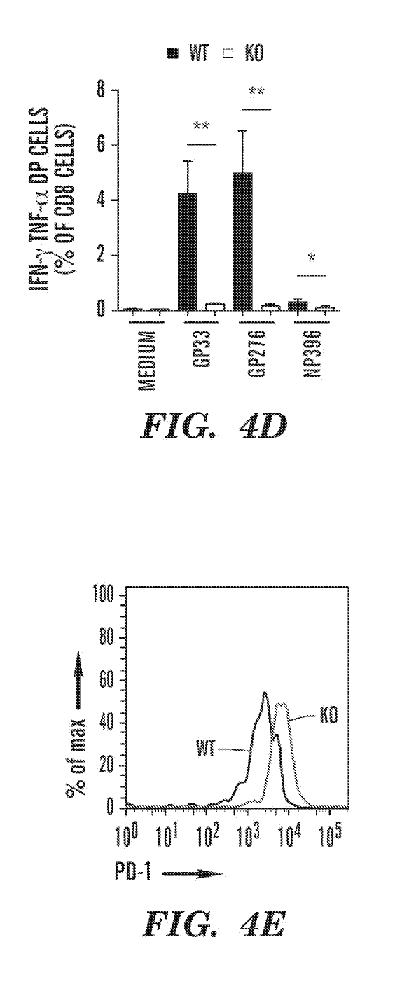

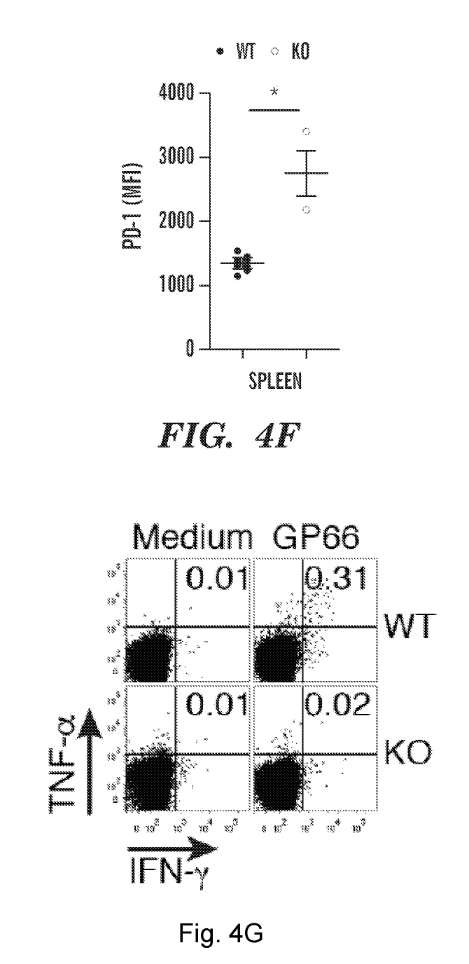

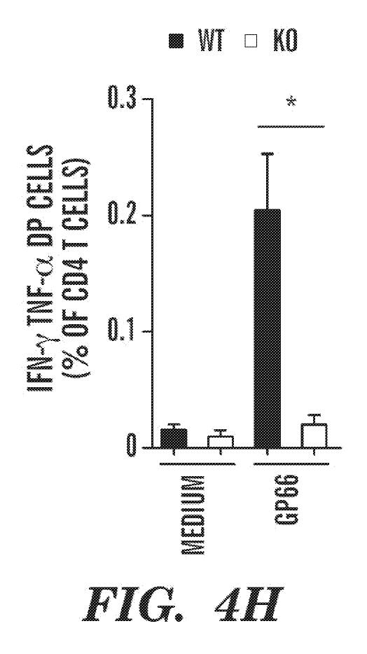

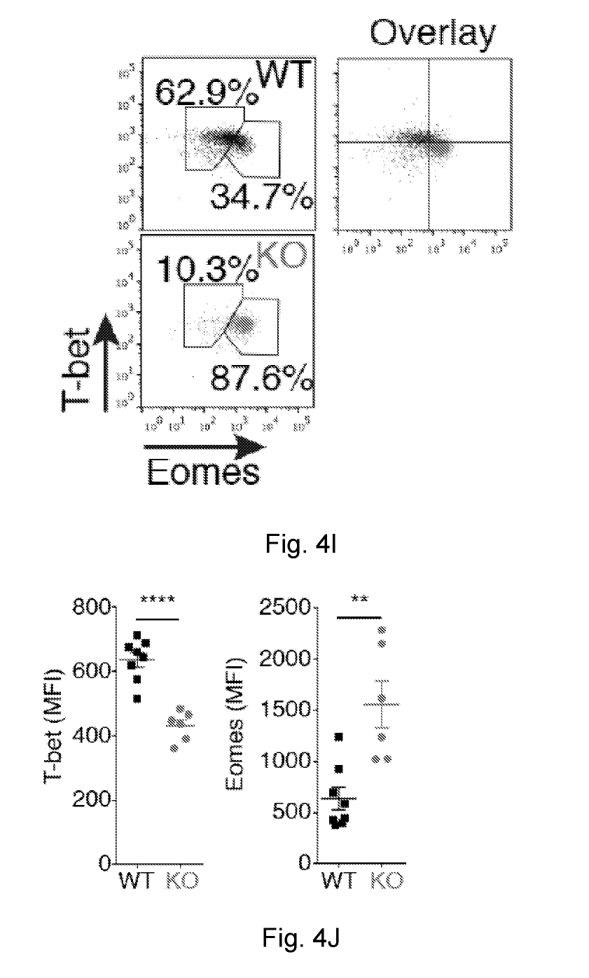

In some embodiments of any of the aspects, administration of the agonist of TMEM16F inhibits or breaks T cell exhaustion (also referred to as T cell tolerance). In one aspect of any of the embodiments, described herein is a method of inhibiting T cell exhaustion, the method comprising contacting a T cell with an agonist of TMEM16F. T cell exhaustion is characterized by compromised, e.g., reduced, effector functions such as cytokine production and cytotoxic activity. T cell exhaustion can also be accompanied by increased expression of inhibitory receptors. Accordingly, inhibition or breaking of T cell exhaustion can be an increase in cytokine production, an increase in cytotoxic activity, an increase in T cell activity, an increase in T cell responsiveness to activation, and/or a decrease in inhibitory receptor expression. Inhibition or breaking of T cell exhaustion can also be a slowing or halt of the rate at which cytokine production, T cell activity, T cell responsiveness and/or cytotoxic activity are decreasing, and/or the rate at which inhibitory receptor expression is increasing. Methods of measuring these parameters are well-known in the art and described in the Examples herein, e.g., the assays depicted in FIGS. 4A-4D.

By way of non-limiting example, in order to determine the effect of an agent on T cell exhaustion, T cell exhaustion can be experimentially induced by contacting T cells with recall antigen, αCD3 in the absence of costimulation, and/or ionomycin. Levels of, e.g. LDH-A, RAB10, and/or ZAP70 (both intracellular or secreted) can be monitored, for example, to determine the extent of T cell exhaustion (with levels of IL-2, IFN-γ and TNFα correlating with increased T cell exhaustion). The response of cells pre-treated with, e.g. ionomycin, to an antigen can also be measured in order to determine the extent of T cell exhaution in a cell or population of cells, e.g. by monitoring the level of secreted and/or intracellular IL-2 and/or TNF-α (see, e.g. Macian et al. Cell 2002 109:719-731; which is incorporated by reference herein in its entirety). Other characteristics of exhausted T cells are that they have increased levels of Fyn and ZAP-70/Syk, Cbl-b, GRAIL, Ikaros, CREM (cAMP response element modulator), B lymphocyte-induced maturation protein-1 (Blimp-1), PD1, CD5, and SHP2; increased phosphorylation of ZAP-70/Syk, LAT, PLCγ1/2, ERK, PKC-θ/IKBA; increased activation of intracellular calcium levels; decreased histone acetylation or hypoacetylation and/or increased CpG methylation at the IL-2 locus. Thus, in some embodiments of any of the aspects, reduction of one or more of any of these parameters can be assayed to determine whether one or more agents or a particular dose of an agent can inhibit or break T cell exhaustion.

Inhibition of T cell exhaustion can also be measured by determining the proliferation of T cells in the presence of a relevant antigen assayed, e.g. by a ³H-thymidine incorporation assay or cell number. Markers of T cell activation after exposure to the relevant antigen can also be assayed, e.g. flow cytometry analysis of cell surface markers indicative of T cell activation (e.g. CD69, CD30, CD25, and HLA-DRs). Reduced T cell activation in response to antigen-challenge is indicative of T cell exhaustion. Conversely, increased T cell activation in response to antigen-challenge is indicative of reduced exhaustion.

Other in vivo models of peripheral tolerance that can be used in some aspects and embodiments to measure inhibition of T cell exhaustion can include, for example, models for peripheral tolerance in which homogeneous populations of T cells from TCR transgenic and double transgenic mice are transferred into hosts that constitutively express the antigen recognized by the transferred T cells, e.g., the H-Y antigen TCR transgenic; pigeon cytochrome C antigen TCR transgenic; or hemagglutinin (HA) TCR transgenic. In such models, T cells expressing the TCR specific for the antigen constitutively or inducibly expressed by the recipient mice typically undergo an immediate expansion and proliferative phase, followed by a period of unresponsiveness, which is reversed when the antigen is removed and/or antigen expression is inhibited. Accordingly, in the presence of an effective dose of a TMEM16F agonist, the T cells in such a model will proliferate or expand, show cytokine activity, etc. significantly more than T cells in the absence of the agonist. Such measurements of proliferation can occur in vivo using T cells labeled with BrDU, CFSE or another intravital dye that allows tracking of proliferation prior to transferring to a recipient animal expressing the antigen, or cytokine reporter T cells, or using ex vivo methods to analyze cellular proliferation and/or cytokine production, such as thymidine proliferation assays, ELISA, cytokine bead assays, and the like.

Reduction of T cell exhaustion can also be assessed by examination of tumor infiltrating lymphocytes or T lymphocytes within lymph nodes that drain from an established tumor. Such T cells exhibit features of exhaustion through expression of cell surface molecules such as PD1, TIM-3 or LAG-3, for example, and decreased secretion of cytokines such as IFN-γ. Accordingly, in the presence of an effective dose of a TMEM16F agonist, increased quantities of T cells with 1) antigen specificity for tumor associated antigens (e.g. as determined by major histocompatibility complex class I or class II tetramers which contain tumor associated peptides) and 2 that are capable of secreting high levels of IFN-γ and cytolytic effector molecules such as granzyme-B will be observed relative to that observed in the absence of the agonist.

In some embodiments of any of the aspects, the methods described herein can increase or improve the immune response of a subject. As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments of any of the aspects, the response is specific for a particular antigen (an “antigen-specific response”), and refers to a response by a CD4 T cell, CD8 T cell, or B cell via their antigen-specific receptor. In some embodiments of any of the aspects, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

Agonists of TMEM16F can be utilized as adjuvants, e.g., to increase the response to a vaccine. In one aspect of any of the embodiments, the methods described herein can relate to a method of administering a vaccine to a subject in need thereof, the method comprising administering a) the vaccine and b) an agonist of TMEM16F to the subject. In some embodiments of any of the aspects, the vaccine and the agonist of TMEM16F are administered sequentially. In some embodiments of any of the aspects, the vaccine and the agonist of TMEM16F are administered concurrently. In some embodiments of any of the aspects, the methods of administering a vaccine described herein can relate to administering the vaccine to a subject with reduced immune function, e.g., an elderly subject, a subject who is immunocompromised, or a subject with a chronic disease (e.g., shingles).

In one aspect of any of the embodiments, described herein is a composition comprising a vaccine and an agonist of TMEM16F. In some embodiments, the vaccine and the agonist of TMEM16F can be formulated in the same solution or lypophilized substance. In one aspect of any of the embodiments, described herein is the combination of a vaccine and an agonist of TMEM16F, e.g., in two separate solutions, lyophilized powders, containers, etc., packaged together for administration to the same patient.

In one aspect of any of the embodiments, described herein is a composition comprising a T cell and an agonist of TMEM16F. In some embodiments, the T cell and the agonist of TMEM16F can be formulated in the same solution or lypophilized substance. In one aspect of any of the embodiments, described herein is the combination of a T cell and an agonist of TMEM16F, e.g., in two separate solutions, lyophilized powders, containers, etc., packaged together for administration to the same patient.

In some embodiments of any of the aspects, the methods described herein can decrease or prevent unresponsiveness or functional exhaustion of the immune system of a subject. As used herein, “unresponsiveness” or “functional exhaustion” with regard to immune cells includes refractivity of immune cells to stimulation, such as stimulation via an activating receptor or a cytokine. Unresponsiveness can occur, for example, because of exposure to immunosuppressants, exposure to high or constant doses of antigen, or through the activity of inhibitor receptors or factors. As used herein, the term “unresponsiveness” includes refractivity to activating receptor-mediated stimulation. Such refractivity is generally antigen-specific and persists after exposure to the antigen has ceased. Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type.

In some embodiments of any of the aspects, administration of the agonist of TMEM16F increases the number and/or proportion of T-bet+ T cells, e.g., in a population of cells or in a subject. In one aspect of any of the embodiments, described herein is a method of increasing T-bet+ T cell activity, proliferation, and/or survival, the method comprising contacting a T cell with an agonist of TMEM16F. As used herein, “T-bet+” or “T-bet^{hi}” refers to any cell, e.g. a T cell, with detectable and/or high expression of T-bet (e.g. human T-bet is NCBI Gene ID: 30009). T-bet+ cells are characterized by being precursor cells, e.g., not terminally differentiated. A high expression of T-bet can be the average level of T-bet found in a population of cells which are not Eomes^{hi} cells. Alternatively, a high expression of T-bet can be the average level of T-bet found in a population of precursor T cells.

In some embodiments of any of the aspects, a subject administered an agonist of TMEM16F can be further administered an immune checkpoint therapy. As used herein “immune checkpoint therapy” refers to a therapy which modulates and/or targets immune checkpoint polypeptides. These polypeptides are checkpoints or key regulators of the human immune response and can inhibit immune activity such as recognition and clearance of tumors by the immune system. Normally, checkpoints prevent damaging over-activation of the immune system. However, some tumors or cancers can promote misregulation of these checkpoints and therefore prevent proper function of the immune system. Suitable targets for immune checkpoint therapies (i.e. exemplary checkpoints) can include, but are not limited to PD-L1 (e.g., human PD-L1, NCBI Gene ID: 29126), PD-1 (e.g., human PD-1, NCBI Gene ID: 5133), CTLA-4 (e.g., human CTLA-4, NCBI Gene ID: 1493), TIM-3 (e.g., human TIM-3, NCBI Gene ID: 84868), and LAG-3 (e.g., human LAG-3, NCBI Gene ID: 3902). In some embodiments of any of the aspects, the immune checkpoint therapy can be, e.g., an anti-PD-1 therapy; an anti-CTLA4 therapy; an anti-PD-L1 therapy, an anti-TIM3 therapy; and/or an anti-LAG3 therapy. Such therapies are known in the art and can include antibody-based therapies, antibody reagents, ligand-mimetic therapies, and/or small molecule therapeutics. By way of non-limiting example, anti-PD-1 therapies can include nivolumab and pembrolizumab; anti-CTLA4 therapies can include ipilimumab; anti-PD-L1 therapies can include atezolizumab; anti-TIM3 therapies can include TSR-022; and anti-LAG3 therapies can include BMS-986016. In some embodiments of any of the aspects, the immune checkpoint therapy can be, e.g., IMP321.

By preventing, reducing, or breaking T cell exhaustion, an agonist of TMEM16F can improve the efficacy of T-cell based therapies, e.g., CAR-T or adoptive cell transfer therapies, by improving and/or prolonging the desired activity of the therapeutic T cell. The therapeutic T cell can be contacted with the agonist of TMEM16F in vivo (e.g., via systemic administration of the agonist of TMEM16F before, during and/or after administration of a therapeutic T cell (e.g. CAR-T) or ex vivo (e.g., before, during, and/or after the process of isolating T cells and/or modifying them to form CAR-Ts).

CAR-T cell and related therapies relate to adoptive cell transfer of immune cells (e.g., T cells) expressing a CAR that binds specifically to a targeted cell type (e.g., cancer cells) to treat a subject. In some embodiments, the cells administered as part of the therapy can be autologous to the subject. In some embodiments, the cells administered as part of the therapy are not autologous to the subject. In some embodiments, the cells are engineered and/or genetically modified to express the CAR. Further discussion of CAR-T therapies can be found, e.g., in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

In some embodiments of any of the aspects, a subject administered an agonist of TMEM16F as described herein is further administered a T cell, e.g., a CAR-T cell. The agonist and the T cell can be administered sequentially and/or concurrently.

In one aspect of any of the embodiments, described herein is a method of administering a CAR-T therapy or adoptive cell transfer therapy to a subject in need thereof, the method comprising a) contacting a CART cell or T cell ex vivo with an agonist of TMEM16F, and b) administering the CAR-T cell or a CAR-T cell obtained from the T cell to the subject. In some embodiments of any of the aspects, the T-cell is contacted with the agonist of TMEM16F prior to modifiying the T cell to create a CAR-T cell. In some embodiments of any of the aspects, the CAR-T is contacted with the agonist of TMEM16F after modifying a T cell to create a CAR-T cell. In some embodiments of any of the aspects, the subject administered a T-cell therapy (e.g., a CAR-T cell) is not administered an agonist of TMEM16F. In some embodiments of any of the aspects, the subject administered a T-cell therapy (e.g., a CAR-T cell) is not administered a separate composition comprising an agonist of TMEM16F and not comprising T-cells. In some embodiments of any of the aspects, the T-cell therapy (e.g., a CAR-T cell), when administered to the subject, does not comprise a detectable level of the agonist of TMEM16F, e.g., the agonist has been removed (e.g., by means of filtration, washing, and/or agonist-binding reagents) or degraded. In some embodiments of any of the aspects, the T-cell (e.g., a CAR-T cell), when administered to the subject, a detectable level of the agonist of TMEM16F is not present in and/or on the cell, e.g., the agonist has been removed (e.g., by means of filtration, washing, and/or agonist-binding reagents) or degraded.

Described herein is the ex vivo use of an agonist of TMEM16F to 1) reduce, prevent, or break T cell exhaustion and/or 2) increases the number and/or proportion of T-bet+ T cells. Such ex vivo manipulation of T cells can have therapeutic applications, e.g., to improve the efficacy of CAR-T therapy as well as research applications, e.g., to improve the activation and/or maintenance of activated T cells for research purposes.

As described herein, modulation of TMEM16F can influence the level and/or activity of PD-1. Inhibition of PD-1 has been implicated in a number of autoimmune and/or inflammatory conditions (see, e.g., International Patent Publication 2013/022091; US Patent Publication 2014/0018252; and Francisco et al. 2010 Immunological Reviews 236:219-242; Dai et al. 2014 Cellular Immunology 290:72-79; Riu et al. 2013 PNAS 110:16073-16078; Reynoso et al. 2009 The Journal of Immunology 182:2102-2112; Siwiec et al. Europe PMC 2015 69:534-542; Kong et al. 2016 Melanoma Research 26:202-204; Heneghan et al. 2013 The Lancet 382:1433-1444; Liu et al. 2015 Arthritis Research and

Therapy 17:340; Dai et al. 2014 Cellular Immunology 290:72-79; Nishimura et al. 2001 Science 291:319-322; Kodogepalli et al. FEBS Letters. 2015; and Laubli et al. 2015 Journal for ImmunoTherapy of Cancer 3:11; each of which is incorporated by reference herein in its entirety. Accordingly, inhibition of TMEM16F can permit the treatment of autoimmune and/or inflammatory conditions, including but not limited to conditions induced by PD-1 inhibiting therapies (e.g., anti-PD-1 antibody therapeutics as discussed elsewhere herein). In one aspect of any of the embodiments, described herein is a method of treating an autoimmune or inflammatory disease in a subject in need thereof, the method comprising administering an inhibitor of TMEM16F to the subject. In some embodiments of any of the aspects, administration of an inhibitor of TMEM16F increases or promotes T cell exhaustion. In one aspect of any of the embodiments, described herein is a method of decreasing the number of T-bet+ T cells in a subject, the method comprising administering an inhibitor of TMEM16F to a subject in need thereof In some embodiments of any of the aspects, the subject is in need of a decrease of an immune response. In some embodiments of any of the aspects, the subject is in need of treatment for an autoimmune disease.

In one aspect of any of the embodiments, described herein is a method of decreasing T-bet+ T cell activity, proliferation, and/or survival, the method comprising contacting a T cell with an inhibitor of TMEM16F. In one aspect of any of the embodiments, described herein is a method of decreasing T cell exhaustion, the method comprising contacting a T cell with an inhibito of TMEM16F. Such ex vivo manipulation of T cells can have research applications, e.g., to prevent or reduce the activation and/or maintenance of activated T cells for research purposes.

As used herein, “autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include cancer cells.

Inhibition of TMEM16F as described herein can promote tolerance or dampen an inappropriate, unwanted, or undesirable immune response, thereby permitting treatment of autoimmune disease and/or conditions associated with transplants (e.g., graft vs. host disease).

Accordingly, in some embodiments of these methods and all such methods described herein, the autoimmune diseases to be treated or prevented using the methods described herein, include, but are not limited to: rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus), gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, and psoriasis. Autoimmune disease has been recognized also to encompass atherosclerosis and Alzheimer's disease.

In some embodiments of the methods of preventing or treating an autoimmune disease, e.g., by promoting T cell tolerance, the subject being administered the inhibitor of TMEM16F as described herein has or has been diagnosed with host versus graft disease (HVGD). In a further such embodiment, the subject being treated with the methods described herein is an organ or tissue transplant recipient. In some embodiments of any of the aspects, the method described herein are used for increasing transplantation tolerance in a subject. In some such embodiments, the subject is a recipient of an allogenic transplant. The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction; inflammation associated with infection; inflammation of the cornea; inflammation of the retina; inflammation of the spinal cord; inflammation associated with organ regeneration; and pulmonary inflammation.

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis.

In some embodiments, a subject in need of treatment for inflammation can be a subject having, or diagnosed as having temporomandibular joint disorders; COPD; smoke-induced lung injury; renal dialysis associated disorders; spinal cord injury; graft vs. host disease; bone marrow transplant or complications thereof; infection; trauma; pain; incisions; surgical incisions; a chronic pain disorder; a chronic bone disorder; mastitis; and joint disease. In some embodiments, trauma can include battle-related injuries or tissue damage occurring during a surgery. Smoke-induced lung injury can result from exposure to tobacco smoke, environmental pollutants (e.g. smog or forest fires), or industrial exposure. By way of non-limiting example, inflammatory conditions can be inflammatory conditions of the skin, such as Sweet's syndrome, pyoderma gangrenosum, subcorneal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxicdermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g.cheilitis, chapped lips, nasal irritation, mucositis and vulvovaginitis).

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, artherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-lassociated myelopathy/tropical spactic paraparesis; Devic's disease; human immunodeficiency virus encephalopathy; human immunodeficiency virus vacuolar myelopathy; peipheral neuropathies; Guillame-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediaterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organising pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g. viral, bacterial, fungal, parasite or prion infections. In some embodiments, an inflammatory condition is associated with an allergic response. In some embodiments, an inflammatory condition is associated with a pollutant (e.g. asbestosis, silicosis, or berylliosis).

In some embodiments, the inflammatory condition can be a local condition, e.g., a rash or allergic reaction.

In some embodiments, the inflammation is associated with a wound. In some embodiments, the technology described herein relates to methods of promoting wound healing. As used herein, “wound” refers broadly to injuries to an organ or tissue of an organism that typically involves division of tissue or rupture of a membrane (e.g., skin), due to external violence, a mechanical agency, or infectious disease. A wound can be an epithelial, endothelial, connective tissue, ocular, or any other kind of wound in which the strength and/or integrity of a tissue has been reduced, e.g. trauma has caused damage to the tissue. The term “wound” encompasses injuries including, but not limited to, lacerations, abrasions, avulsions, cuts, burns, velocity wounds (e.g., gunshot wounds), penetration wounds, puncture wounds, contusions, diabetic wounds, hematomas, tearing wounds, and/or crushing injuries. In one aspect, the term “wound” refers to an injury to the skin and subcutaneous tissue initiated in any one of a variety of ways (e.g., pressure sores from extended bed rest, wounds induced by trauma, cuts, ulcers, burns and the like) and with varying characteristics. As used herein, the term “wound healing” refers to a process by which the body of a wounded organism initiates repair of a tissue at the wound site (e.g., skin). The wounds healing process requires, in part, angiogenesis and revascularization of the wounded tissue. Wound healing can be measured by assessing such parameters as contraction, area of the wound, percent closure, percent closure rate, and/or infiltration of blood vessels as known to those of skill in the art. In some embodiments, the particles and compositions described herein can be applied topically to promote wound healing.

In some embodiments of any of the aspects, the autoimmune or inflammatory disease treated according to the methods described herein is inflammatory bowel disease; type I diabetes; multiple sclerosis; Systemic lupus erythematosus (SLE); Crohn's disease; autoimmune dilated cardiomyopathy; autoimmune myocarditis; autoimmune enteritis; arthritis; rheumatoid arthritis; collagen-induced arthritis; autoimmune hemolytic anemia; or autoimmune hepatitis.

In some embodiments of any of the aspects, the subject treated with an inhibitor of TMEM16F according to the methods described herein is a subject receiving or who has received an anti-PD-1 therapy, e.g., a subject who has developed an autoimmune or inflammatory condition due to the anti-PD-1 therapy. In some embodiments of any of the aspects, the subject treated with an inhibitor of TMEM16F according to the methods described herein is a subject receiving or who has received a checkpoint inhibitor therapy, e.g., a subject who has developed an autoimmune or inflammatory condition due to the checkpoint inhibitory therapy. In some embodiments of any of the aspects, the subject treated with an inhibitor of TMEM16F according to the methods described herein is a subject with reduced levels and/or activity of PD-1 as compared to a healthy subject and/or a subject who has not received an anti-PD-1 therapy. In some embodiments of any of the aspects, the subject treated with an inhibitor of TMEM16F according to the methods described herein is a subject with reduced levels and/or activity of PD-L1 as compared to a healthy subject.

As used herein, the term “inhibitor” refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, TMEM16F, e.g. its ability to decrease the level and/or activity of TMEM16F, can be determined, e.g. by measuring the level of an expression product of TMEM16F and/or the activity of TMEM16F. In some embodiments, the inhibitor can be an inhibitory nucleic acid; an aptamer; an antibody reagent; an antibody; or a small molecule.

In some embodiments, an inhibitor of a polypeptide can be an antibody reagent specific for that polypeptide. In some embodiments, a TMEM16F inhibitor can be an anti-TMEM16F antibody reagent.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to TMEM16F.

Inhibitors of the expression of a given gene can be an inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of a target, e.g. TMEM16F. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6, 239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH2-NH—CH2-, —CH2-N(CH3)—O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2).nOCH3, O(CH2)nNH2, O(CH2) nCH3, O(CH2)nONH2, and O(CH2)nONRCH2)nCH3)]2, where n and m are from 1 to about 10. In other embodiments, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

The term “aptamer” refers to a nucleic acid molecule that is capable of binding to a target molecule, such as a polypeptide. For example, an aptamer of the invention can specifically bind to a target molecule, or to a molecule in a signaling pathway that modulates the expression and/or activity of a target molecule. The generation and therapeutic use of aptamers are well established in the art. See, e.g., U.S. Pat. No. 5,475,096.

In some embodiments of any of the aspects, the methods described herein relate to treating a subject having or diagnosed as having, e.g., cancer or a chronic infection with a TMEM16F agonist. Subjects having such conditions can be identified by a physician using current methods of diagnosing them. For example, symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer or exposure to risk factors for cancer (e.g. smoking or radiation) can also aid in determining if a subject is likely to have cancer or in making a diagnosis of cancer.

The compositions and methods described herein can be administered to a subject having or diagnosed as having, e.g., cancer or a chronic infection. In some embodiments of any of the aspects, the methods described herein comprise administering an effective amount of compositions described herein, e.g. a TMEM16F to a subject in order to alleviate a symptom of, e.g., a cancer or chronic infection. As used herein, “alleviating a symptom” of a condition is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of an agonist of TMEM16F needed to alleviate at least one or more symptom of the disease or disorder, and relates to a sufficient amount of pharmacological composition to provide the desired effect. The term “therapeutically effective amount” therefore refers to an amount of a TMEM16F agonist that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active agent which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for T cell exhaustion as described herein, among others. The dosage can be determined by a physician and adjusted, as necessary, to suit observed effects of the treatment.

In one aspect of any of the embodiments, described herein is an agonist of TMEM16F for use in the treatment of a viral infection or cancer in a subject in need thereof or increasing the number of T-bet+ T cells in a subject in need thereof.

In one aspect of any of the embodiments, described herein is the combination of an agonist of TMEM16F and another cancer treatment for use in the treatment of cancer in a subject in need thereof. In one aspect of any of the embodiemnts, described herein is the combination of a) an agonist of TMEM16F and b) another cancer treatment, an immune checkpoint therapy, and/or a CAR-T cell for use in the treatment of cancer in a subject in need thereof. In one aspect of any of the the embodiments, described herein is the combination of a) a vaccine and b) an agonist of TMEM16F for use in administering a vaccine to a subject in need thereof. Combinations of two or more agents can be provided as a single composition (e.g., a solution, suspension, lyophilisate, or the like) comprising each of the two or more agents, e.g., in a container suitable for administration of the composition to a subject (e.g., a syringe, ampoule, or vial). Combinations of two or more agents can also be provided as a seperate compositions present in the same package or kit for administration to the same subject, e.g., simultaneously or concurrently. The separate compositions can be administered separately, or combined prior to administration to the subject.

In some embodiments of any of the aspects, the technology described herein relates to a pharmaceutical composition comprising a TMEM16F agonist as described herein, and optionally a pharmaceutically acceptable carrier. In some embodiments of any of the aspects, the active ingredients of the pharmaceutical composition comprise a TMEM16F agonist as described herein. In some embodiments of any of the aspects, the active ingredients of the pharmaceutical composition consist essentially of a TMEM16F agonist as described herein. In some embodiments of any of the aspects, the active ingredients of the pharmaceutical composition consist of a TMEM16F agonist as described herein. In some embodiments of any of the aspects, the pharmaceutical composition can further comprise a further agent as described herein, e.g., a CAR-T cell, a vaccine, an immune checkpoint therapy, or another cancer therapeutic.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent, e.g. a TMEM16F agonist as described herein.

In some embodiments of any of the aspects, the pharmaceutical composition comprising a TMEM16F agonist as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a TMEM16F agonist as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agent as disclosed herein can also be incorporated into the parenteral dosage forms of the disclosure, including conventional and controlled-release parenteral dosage forms.

Pharmaceutical compositions comprising a TMEM16F agonist can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the TMEM16F agonist can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos.: 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1 ; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment for cancer can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE.®. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.®.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

In certain embodiments, an effective dose of a composition comprising a TMEM16F agonist as described herein can be administered to a patient once. In certain embodiments, an effective dose of a composition comprising a TMEM16F agonist can be administered to a patient repeatedly. For systemic administration, subjects can be administered a therapeutic amount of a composition comprising a TMEM16F agonist, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a TMEM16F agonist can be administered over a period of time, such as over a 5 minute, 10 minute, 15 minute, 20 minute, or 25 minute period.

The dosage ranges for the administration of a TMEM16F agonist according to the methods described herein depend upon, for example, the form of the agonist, its potency, and the extent to which symptoms, markers, or indicators of a condition described herein are desired to be reduced, for example the percentage reduction desired for tumor size or T cell exhaustion or the extent to which, for example, an immune response is desired to be induced. The dosage should not be so large as to cause adverse side effects, such as autoimmune conditions. Generally, the dosage will vary with the age, condition, and sex of the patient and can be determined by one of skill in the art. The dosage can also be adjusted by the individual physician in the event of any complication.

The efficacy of a TMEM16F agonist in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. T cell responses or exhaustion. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. T cell responses/activity). It is well within the ability of one skilled in the art to monitor efficacy of administration and/or treatment by measuring any one of such parameters, or any combination of parameters. Efficacy can be assessed in animal models of a condition described herein, for example treatment of cancer or infection models. When using an experimental animal model, efficacy of treatment is evidenced when a statistically significant change in a marker is observed, e.g. a measure of T cell activity/exhaustion.

Provided herein are multiple assays and methods for measuring the activity of TMEM16F. It is contemplated that these assays and methods can also be used to identify modulators of TMEM16F or measure the activity of such modulators. The modulators can be, e.g., agonists or inhibitors and can modulate the level and/or activity of TMEM16F.

Provided herein are methods and assays for measuring the activity of a TMEM16F agonist and/or a candidate TMEM16F agonist. In some embodiments of any of the aspects, the methods and assays can relate to identifying a TMEM16F agonist and/or identifying a TMEM16F agonist candidate (e.g., a candidate agent) as a TMEM16F agonist.

As described herein, TMEM16F possesses scramblase activity, particularly with respect to PS. Thus, TMEM16F can move PS from the inner surface of a membrane to the outer surface of a membrane or vice versa. This movement of PS, or the relative movement of PS in two samples, can be detected in order to determine the activity of TMEM16F, e.g., in the presence or absence of a given agent.

In one aspect of any of the embodiments, described herein is a method of measuring the activity of a TMEM16F agonist candidate, the method comprising: contacting one side of a membrane comprising TMEM16F and NBD-phospholipids with dithionite; contacting TMEM16F with the agonist candidate; and measuring the fluorescence of the NBD-phospholipids; wherein the greater the decrease in the fluorescence, the greater the TMEM16F agonist activity of the agonist candidate. NBD-phospholipids are phospholipids fluoresencently labelled with 7-nitrobenz-2-oxa-1,3-diazol-4-yl (NBD). The fluorescence of NBD is quenched in the presence of ditihionite. Accordingly, in the foregoing assay, if a NBD label is exposed on the surface of the membrane where dithionite is present, the fluorescence will be quenched. In the presence of active TMEM16F, the NBD-phospholipids will be more likely to be moved from one surface to another, resulting in each NBD-phopholipid being more likely to be quenched over time. As TMEM16F activity increases, the amount and/or rate of quenching will increase and the detectable fluorescence signal will decrease. In some embodiments of any of the aspects, a NBD-phospholipid assay for TMEM16F activity can be an in vitro assay. In some embodiments of any of the aspects, a NBD-phospholipid assay for TMEM16F activity can be a non-cellular assay, e.g., functional cells are not present in the assay. In some embodiments of any of the aspects, a NBD-phospholipid can be a NBD-PS molecule.

The ability of TMEM16F to move PS from one surface of a membrane to another can also be monitored by measuring the binding of annexinV (e.g., a fluoresecently tagged annexinV) to PS. Such fluoresecently-labelled reagents are readily available, e.g., Cat Nos. A23202, A13201, A35108, A13202, A13203, A23204, and A35109 from ThermoFischer; Waltham, Mass. In one aspect of any of the embodiments, described herein is a method of measuring the activity of a TMEM16F agonist candidate, the method comprising: contacting a membrane comprising TMEM16F with the agonist candidate; contacting one surface of the membrane with fluoresecently-labelled annexinV; measuring the level of annexinV staining on the membrane surface; wherein the greater the level of staining, the greater the activity of the agonist candidate. In one aspect of any of the embodiments, described herein is a method of measuring the activity of a TMEM16F agonist candidate, the method comprising: contacting a cell comprising TMEM16F with the agonist candidate; contacting the cell with fluoresecently-labelled annexinV; measuring the level of annexinV staining on the cell surface; wherein the greater the level of staining, the greater the activity of the agonist candidate.

In some embodiments of any of the aspects, the membrane is a phospholipid membrane. In some embodiments of any of the aspects, the membrane is a phospholipid bilayer membrane. In some embodiments of any of the aspects, the membrane is biological in origin or mimics the content of a biological membrane. In some embodiments of any of the aspects, the membrane forms and/or is a liposome. In some embodiments of any of the aspects, the membrane does not comprise another scramblase.

Calcium ionophores, e.g., A12387, can induce TMEM16F activiation. In some embodiments of any of the aspects, a step of contacting TMEM16F (or a membrane or cell comprising TMEM16F) with an agonist or agonist candidate can further comprise contacting TMEM16F, or the cell or membrane, with a calcium ionophore. In some embodiemnts of any of the aspects, the calcium ionophore can be A12387.

In some embodiments of any of the aspects, fluoresence can be measured by fluoresence microscopy. In some embodiments of any of the aspects, fluoresence can be measured by flow cytometry.

In some embodiments of any of the aspects, the NBD-phospholipid assay for TMEM16F activity can be used as a primary screen to identify TMEM16F agonists. In some embodiments of any of the aspects, the annexinV assay for TMEM16F activity can be used as a secondary screen to identify TMEM16F agonists.

Any of the foregoing assays for measuring the activity of TMEM16F agonists or agonist candidates can be applied to measuring the activity of TMEM16F inhibitors or inhibitor candidates, e.g., wherein the opposite effect of that indicating agonist activity indicates inhibitor activity.

As used herein, the terms “candidate compound” or “candidate agent” refer to a compound or agent and/or compositions thereof that are to be screened for their ability to, e.g., increase the level and/or activity of TMEM16F. Candidate compounds and/or agents can be produced recombinantly using methods well known to those of skill in the art (see Sambrook et al., Molecular Cloning: A Laboratory Manual (2 ed.), Cold Spring Harbor Laboratory Press, Cold Spring Harbor, N.Y., USA (1989)) or synthesized. Candidate compounds and agents can be screened for their ability to increase the level and/or activity of TMEM16F, e.g. in vitro or in vivo. In one embodiment, candidate agents are screened using the assays described above herein. Candidate agents are typically first screened for activity in vitro and those candidate agents with activity are identified. In vivo assays can then be conducted on the identified agents.

As used herein, the terms “compound” or “agent” are used interchangeably and refer to molecules and/or compositions including, but not limited to chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies and intrabodies, or fragments thereof.

Compounds can be tested at any concentration that can modulate expression or protein activity relative to a control over an appropriate time period. In some embodiments of any of the aspects, compounds are tested at concentrations in the range of about 0.1 nM to about 1000 mM. In one embodiment, the compound is tested in the range of about 0.1 μM to about 20 μM, about 0.1 μM to about 10 μM, or about 0.1 μM to about 5 μM. In one embodiment, compounds are tested at 1 μM. Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds may be screened individually, or in groups. Group screening is particularly useful where hit rates for effective test compounds are expected to be low such that one would not expect more than one positive result for a given group.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer and/or chronic infection. A subject can be male or female.

A subject can be one who has been previously diagnosed with or identified as suffering from or having a condition in need of treatment (e.g. cancer and/or chronic infection) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, “contacting” refers to any suitable means for delivering, or exposing, an agent to at least one complex, enzyme, or cell. Exemplary delivery methods include, but are not limited to, direct delivery to cell culture medium, perfusion, injection, or other delivery method well known to one skilled in the art.

As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. scramblase activity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments of any of the aspects, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments of any of the aspects, the polypeptide described herein can be a variant of a sequence described herein. In some embodiments of any of the aspects, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, a nucleic acid encoding a polypeptide as described herein (e.g. a TMEM16F polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments of any of the aspects, be combined with other suitable compositions and therapies. In some embodiments of any of the aspects, the vector is episomal. The use of a suitable episomal vector provides a means of maintaining the nucleotide of interest in the subject in high copy number extra chromosomal DNA thereby eliminating potential effects of chromosomal integration.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. Accordingly, as used herein, “selectively binds” or “specifically binds” refers to the ability of an antibody reagent (e.g., an antibody or portion thereof) to bind to a target, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a cancer or chronic infection. The term “treating” includes reducing or alleviating at least one adverse effect or symptom of a condition, disease or disorder associated with, e.g., a cancer or chronic infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating a viral infection or cancer in a subject in need thereof, the method comprising administering an agonist of TMEM16F to the subject.
2. The method of paragraph 1, whereby T cell exhaustion is inhibited.
3. A method of increasing the number of T-bet+ T cells in a subject, the method comprising administering an agonist of TMEM16F to a subject in need thereof
4. The method of paragraph 3, wherein the subject is in need of an increase of an immune response to a chronic disease.
5. The method of any of paragraphs 3-4, wherein the subject is in need of treatment for a viral infection or cancer.
6. The method of paragraph 5, wherein the viral infection is an HIV or hepatitis B virus (HBV).
7. The method of paragraph 5, wherein the cancer is a recurrent cancer.
8. The method of paragraphs 5 or 7, wherein the agonist of TMEM16F is administered before another cancer treatment.
9. The method of paragraphs 5 or 7-8, wherein the agonist of TMEM16F is administered concurrently with another cancer treatment.
10. The method of paragraph 5, wherein the cancer is selected from the group consisting of:
   melanoma; NSCLC; renal cell carcinoma; Hodgkin lymphoma; and bladder cancer.
11. The method of any of paragraphs 1-10, wherein the subject is further administered an immune checkpoint therapy.
12. The method of paragraph 11, wherein the immune checkpoint therapy is selected from the group consisting of:
   an anti-PD-1 therapy; an anti-CTLA4 therapy; an anti-PD-L1 therapy; an anti-TIM-3 therapy; and an anti-LAG-3 therapy.
13. The method of paragraph 11, wherein the anti-PD-1 therapy is selected from the group consisting of:
   nivolumab; and pembrolizumab.
14. The method of paragraph 11, wherein the anti-CTLA4 therapy is ipilimumab.
15. The method of paragraph 11, wherein the anti-PD-L1 therapy is atezolizumab.
16. The method of paragraph 11, wherein the anti-TIM3 therapy is TSR-022.
17. The method of paragraph 11, wherein the anti-LAG3 therapy is BMS-986016.
18. The method of any of paragraphs 1-17, wherein the subject is further administered a CAR-T cell.
19. A method of administering a vaccine to a subject in need thereof, the method comprising administering:
   the vaccine;
   and an agonist of TMEM16F to the subject.
20. The method of paragraph 19, wherein the vaccine and the agonist of TMEM16F are administered sequentially.
21. The method of paragraph 19, wherein the vaccine and the agonist of TMEM16F are administered concurrently.
22. The method of any of paragraphs 19-21, wherein the subject is a subject with reduced immune function.
23. The method of paragraph 22, wherein the subject with reduced immune function is a subject with a chronic disease.
24. A composition comprising:
   a vaccine; and an agonist of TMEM16F.
25. A method of administering a CAR-T therapy to a subject in need thereof, the method comprising: contacting a CAR-T cell or T cell ex vivo with an agonist of TMEM16F; and
   administering the CAR-T cell or a CAR-T cell obtained from the T cell to the subject.
26. The method of paragraph 26, wherein the T-cell is contacted with the agonist of TMEM16F prior to modifiying the T cell to create a CAR-T cell.
27. The method of any of paragraphs 26-27, wherein the CAR-T is contacted with the agonist of TMEM16F after modifying a T cell to create a CAR-T cell.
28. The method of any of paragraphs 26-28, wherein the subject is not administered an agonist of TMEM16F.
29. The method of any of paragraphs 26-29, wherein a detectable level of the agonist of TMEM16F is not present in or on the CAR-T cell when the CAR-T cell is administered to the subject.
30. A method of increasing T-bet+ T cell activity, proliferation, and/or survival, the method comprising contacting a T cell with an agonist of TMEM16F.
31. A method of inhibiting T cell exhaustion, the method comprising contacting a T cell with an agonist of TMEM16F.
32. The method of any of paragraphs 30-31, wherein the T cell is a T cell obtained from a subject or a T cell differentiated from a cell obtained from a subject.
33. The method of any of paragraphs 30-32, wherein the T cell is administered to a subject after the contacting step.
34. A method of measuring the activity of a TMEM16F agonist candidate, the method comprising:
   contacting a membrane comprising TMEM16F and NBD-phospholipids with dithionite and the agonist candidate; and
   measuring the fluorescence of the NBD-phospholipids;
   wherein the greater the decrease in the fluorescence, the greater the activity of the agonist candidate.
35. The method of paragraph 34, wherein the membrane is a liposome.
36. The method of any of paragraphs 34-35, wherein the membrane does not comprise another scramblase.
37. A method of measuring the activity of a TMEM16F agonist candidate, the method comprising:
   contacting a cell with the agonist candidate;
   contacting the cell with annexinV; and
   measuring the level of annexinV staining on the cell surface;
   wherein the greater the level of staining, the greater the activity of the agonist candidate.
38. The method of paragraph 37, wherein the cell wherein the first contacting step further comprises contacting the cell with the calcium ionophore A23187.
39. The method of any of paragraphs 1-38, wherein the agonist of TMEM16F is an SSRI inhibitor.
40. A method of treating an autoimmune or inflammatory disease in a subject in need thereof, the method comprising administering an inhibitor of TMEM16F to the subject.
41. The method of paragraph 40, whereby T cell exhaustion is increased.
42. A method of decreasing the number of T-bet+ T cells in a subject, the method comprising administering an inhibitor of TMEM16F to a subject in need thereof.
43. The method of paragraph 42, wherein the subject is in need of a decrease of an immune response.
44. The method of paragraph 42, wherein the subject is in need of treatment for an autoimmune or inflammatory disease.
45. The method of any of paragraphs 40-44, wherein the autoimmune or inflammatory disease is selected from the group consisting of:
   inflammatory bowel disease,; type I diabetes; multiple sclerosis; Systemic lupus erythematosus (SLE); Crohn's disease; autoimmune dilated cardiomyopathy; autoimmune myocarditis; autoimmune enteritis; arthritis; rheumatoid arthritis; collagen-induced arthritis; autoimmune hemolytic anemia; autoimmune hepatitis.
46. A method of decreasing T-bet+ T cell activity, proliferation, and/or survival, the method comprising contacting a T cell with an inhibitor of TMEM16F.
47. A method of decreasing T cell exhaustion, the method comprising contacting a T cell with an inhibitor of TMEM16F.
48. The method of any of paragraphs 40-47, wherein the inhibitor of TMEM16F is an inhibitory nucleic acid; an aptamer; an antibody reagent; an antibody; or a small molecule.

Some embodiments of the technology described herein can be defined according to any of the following numbered paragraphs:
1. A method of treating a viral infection or cancer in a subject in need thereof, the method comprising administering an agonist of TMEM16F to the subject.
2. The method of paragraph 1, whereby T cell exhaustion is inhibited.
3. A method of increasing the number of T-bet+ T cells in a subject, the method comprising administering an agonist of TMEM16F to a subject in need thereof.
4. The method of paragraph 3, wherein the subject is in need of an increase of an immune response to a chronic disease.
5. The method of any of paragraphs 3-4, wherein the subject is in need of treatment for a viral infection or cancer.
6. The method of paragraph 5, wherein the viral infection is an HIV or hepatitis B virus (HBV).
7. The method of paragraph 5, wherein the cancer is a recurrent cancer.
8. The method of paragraphs 5 or 7, wherein the agonist of TMEM16F is administered before another cancer treatment.
9. The method of paragraphs 5 or 7-8, wherein the agonist of TMEM16F is administered concurrently with another cancer treatment.
10. The method of paragraph 5, wherein the cancer is selected from the group consisting of:
   melanoma; NSCLC; renal cell carcinoma; Hodgkin lymphoma; and bladder cancer.
11. The method of any of paragraphs 1-10, wherein the subject is further administered an immune checkpoint therapy.
12. The method of paragraph 11, wherein the immune checkpoint therapy is selected from the group consisting of:
   an anti-PD-1 therapy; an anti-CTLA4 therapy; an anti-PD-L1 therapy; an anti-TIM-3 therapy; and an anti-LAG-3 therapy.
13. The method of paragraph 11, wherein the anti-PD-1 therapy is selected from the group consisting of:
   nivolumab; and pembrolizumab.
14. The method of paragraph 11, wherein the anti-CTLA4 therapy is ipilimumab.
15. The method of paragraph 11, wherein the anti-PD-L1 therapy is atezolizumab.
16. The method of paragraph 11, wherein the anti-TIM3 therapy is TSR-022.
17. The method of paragraph 11, wherein the anti-LAG3 therapy is BMS-986016.
18. The method of any of paragraphs 1-17, wherein the subject is further administered a CAR-T cell.
19. A method of administering a vaccine to a subject in need thereof, the method comprising administering:
   the vaccine;
   and an agonist of TMEM16F to the subject.
20. The method of paragraph 19, wherein the vaccine and the agonist of TMEM16F are administered sequentially.
21. The method of paragraph 19, wherein the vaccine and the agonist of TMEM16F are administered concurrently.
22. The method of any of paragraphs 19-21, wherein the subject is a subject with reduced immune function.
23. The method of paragraph 22, wherein the subject with reduced immune function is a subject with a chronic disease.
24. A composition comprising:
   a vaccine; and an agonist of TMEM16F.
25. A method of administering a CAR-T therapy to a subject in need thereof, the method comprising:
   contacting a CAR-T cell or T cell ex vivo with an agonist of TMEM16F; and
   administering the CAR-T cell or a CAR-T cell obtained from the T cell to the subject.
26. The method of paragraph 26, wherein the T-cell is contacted with the agonist of TMEM16F prior to modifiying the T cell to create a CAR-T cell.
27. The method of any of paragraphs 26-27, wherein the CAR-T is contacted with the agonist of TMEM16F after modifying a T cell to create a CAR-T cell.
28. The method of any of paragraphs 26-28, wherein the subject is not administered an agonist of TMEM16F.
29. The method of any of paragraphs 26-29, wherein a detectable level of the agonist of TMEM16F is not present in or on the CAR-T cell when the CAR-T cell is administered to the subject.
30. A method of increasing T-bet+ T cell activity, proliferation, and/or survival, the method comprising contacting a T cell with an agonist of TMEM16F.
31. A method of inhibiting T cell exhaustion, the method comprising contacting a T cell with an agonist of TMEM16F.
32. The method of any of paragraphs 30-31, wherein the T cell is a T cell obtained from a subject or a T cell differentiated from a cell obtained from a subject.
33. The method of any of paragraphs 30-32, wherein the T cell is administered to a subject after the contacting step.
34. A method of measuring the activity of a TMEM16F agonist candidate, the method comprising:
   contacting a membrane comprising TMEM16F and NBD-phospholipids with dithionite and the agonist candidate; and
   measuring the fluorescence of the NBD-phospholipids;
   wherein the greater the decrease in the fluorescence, the greater the activity of the agonist candidate.
35. The method of paragraph 34, wherein the membrane is a liposome.
36. The method of any of paragraphs 34-35, wherein the membrane does not comprise another scramblase.
37. A method of measuring the activity of a TMEM16F agonist candidate, the method comprising:
   contacting a cell with the agonist candidate;
   contacting the cell with annexinV; and
   measuring the level of annexinV staining on the cell surface;
   wherein the greater the level of staining, the greater the activity of the agonist candidate.
38. The method of paragraph 37, wherein the cell wherein the first contacting step further comprises contacting the cell with the calcium ionophore A23187.
39. The method or composition of any of paragraphs 1-38, wherein the agonist of TMEM16F is a TMEM16F polypeptide, a nucleic acid encoding a TMEM16F polypeptide, or an SSRI inhibitor.
40. The method or composition of paragraph 39, wherein the SSRI inhibitor is fluoxetine, sertraline, paroxetine, citalopram, escitalopram, fluvoxamine, dapoxetine, indalpine, zimelidine, cericlamine, or panuramine.
41. A method of treating an autoimmune or inflammatory disease in a subject in need thereof, the method comprising administering an inhibitor of TMEM16F to the subject.
42. The method of paragraph 41, whereby T cell exhaustion is increased.
43. A method of decreasing the number of T-bet+ T cells in a subject, the method comprising administering an inhibitor of TMEM16F to a subject in need thereof.
44. The method of paragraph 43, wherein the subject is in need of a decrease of an immune response.
45. The method of paragraph 43, wherein the subject is in need of treatment for an autoimmune or inflammatory disease.
46. The method of any of paragraphs 41-45, wherein the autoimmune or inflammatory disease is selected from the group consisting of:
   inflammatory bowel disease,; type I diabetes; multiple sclerosis; Systemic lupus erythematosus (SLE); Crohn's disease; autoimmune dilated cardiomyopathy; autoimmune myocarditis; autoimmune enteritis; arthritis; rheumatoid arthritis; collagen-induced arthritis; autoimmune hemolytic anemia; autoimmune hepatitis.
47. A method of decreasing T-bet+ T cell activity, proliferation, and/or survival, the method comprising contacting a T cell with an inhibitor of TMEM16F.
48. A method of decreasing T cell exhaustion, the method comprising contacting a T cell with an inhibitor of TMEM16F.
49. The method of any of paragraphs 41-48, wherein the inhibitor of TMEM16F is an inhibitory nucleic acid; an aptamer; an antibody reagent; an antibody; or a small molecule.
50. An agonist of TMEM16F for use in the treatment of a viral infection or cancer in a subject in need thereof.
51. An agonist of TMEM16F for use in increasing the number of T-bet+ T cells in a subject in need thereof.
52. The agonist of TMEM16F of paragraph 51, wherein the subject is in need of an increase of an immune response to a chronic disease.
53. The agonist of any of paragraphs 51-52, wherein the subject is in need of treatment for a viral infection or cancer.
54. The agonist of paragraph 50 or 53, wherein the viral infection is an HIV or hepatitis B virus (HBV).
55. The combination of an agonist of TMEM16F and another cancer treatment for use in the treatment of cancer in a subject in need thereof.
56. The combination of a) an agonist of TMEM16F and b) another cancer treatment, an immune checkpoint therapy, and/or a CAR-T cell for use in the treatment of cancer in a subject in need thereof.
57. The agonist or combination of any of paragraphs 50, 53, 55, or 56, wherein the cancer is a recurrent cancer.
58. The agonist or combination of any of paragraphs 50, 53, or 55-57, wherein the cancer is selected from the group consisting of:
   melanoma; NSCLC; renal cell carcinoma; Hodgkin lymphoma; and bladder cancer.
59. The combination of any of paragraphs 56-58, wherein the immune checkpoint therapy is selected from the group consisting of:
   an anti-PD-1 therapy; an anti-CTLA4 therapy; an anti-PD-L1 therapy; an anti-TIM-3 therapy; and an anti-LAG-3 therapy.
60. The combination of paragraph 59, wherein the anti-PD-1 therapy is selected from the group consisting of:
   nivolumab; and pembrolizumab.
61. The combination of paragraph 59, wherein the anti-CTLA4 therapy is ipilimumab.
62. The combination of paragraph 59, wherein the anti-PD-L1 therapy is atezolizumab.
63. The combination of paragraph 59, wherein the anti-TIM3 therapy is TSR-022.
64. The combination of paragraph 59, wherein the anti-LAG3 therapy is BMS-986016.
65. The combination of any of paragraphs 55-64, wherein a) the agonist and b) another cancer treatment, an immune checkpoint therapy, and/or a CAR-T cell are present in the same composition.
66. The combination of any of paragraphs 55-64, wherein a) the agonist and b) another cancer treatment, an immune checkpoint therapy, and/or a CAR-T cell are present in the same packaging.
67. The combination of a) a vaccine and b) an agonist of TMEM16F for use in administering a vaccine to a subject in need thereof.
68. The combination of paragraph 67, wherein the vaccine and the agonist are present in the same composition.
69. The combination of paragraph 67, wherein the vaccine and the agonist are present in the same packaging.
70. The combination of any of paragraphs 67-69, wherein the subject is a subject with reduced immune function.
71. The combination of paragraph 70, wherein the subject with reduced immune function is a subject with a chronic disease.
72. The agonist or combination of any of paragraphs 50-71, wherein the agonist of TMEM16F is a TMEM16F polypeptide, a nucleic acid encoding a TMEM16F polypeptide, or an SSRI inhibitor.
73. The agonist or combination of paragraph 72, wherein the SSRI inhibitor is fluoxetine, sertraline, paroxetine, citalopram, escitalopram, fluvoxamine, dapoxetine, indalpine, zimelidine, cericlamine, or panuramine.
74. An inhibitor of TMEM16F for use in the treatment of an autoimmune or inflammatory disease in a subject in need thereof.
75. An inhibitor of TMEM16F for use in decreasing the number of T-bet+ T cells in a subject in need thereof.
76. The inhibitor of paragraph 75, wherein the subject is in need of a decrease of an immune response.
77. The inhibitor of paragraph 76, wherein the subject is in need of treatment for an autoimmune or inflammatory disease.
78. The inhibitor of any of paragraphs 74-77, wherein the autoimmune or inflammatory disease is selected from the group consisting of:
   inflammatory bowel disease,; type I diabetes; multiple sclerosis; Systemic lupus erythematosus (SLE); Crohn's disease; autoimmune dilated cardiomyopathy; autoimmune myocarditis; autoimmune enteritis; arthritis; rheumatoid arthritis; collagen-induced arthritis; autoimmune hemolytic anemia; autoimmune hepatitis.
79. The inhibitor of any of paragraphs 74-78, wherein the inhibitor of TMEM16F is an inhibitory nucleic acid; an aptamer; an antibody reagent; an antibody; or a small molecule.

### EXAMPLES

### Example 1

### Scramblase TMEM16F Terminates T Cell Receptor Signaling to Restrict T Cell Exhaustion

In chronic infection, T cells become hyporesponsive to antigenic stimulation to prevent immunopathology. It is demonstrated herein that TMEM16F is required to curb excessive T cell responses in chronic infection with virus. TMEM16F-deficient T cells are hyperactivated during the early phase of infection, exhibiting increased proliferation and cytokine production. Interestingly, this overactivation ultimately leads to severe T cell exhaustion and the inability of the host to control viral burden. Mechanistically, TMEM16F is identified herein as the dominant lipid scramblase in T lymphocytes that transports phospholipids across membranes. TMEM16F is located in late endosomes, where it facilitates the generation of multivesicular bodies (MVBs) for T cell receptor (TCR) degradation and signal termination. Consequently, TMEM16F deficiency results in sustained signaling and augmented T cell activation. The results presented herein demonstrate that scramblase restricts TCR responses to avoid overactivation, ensuring a well-balanced immune response in chronic infectious disease.

T cell activation is central to the adaptive immune response (Smith-Garvin et al., 2009). It occurs after recognition of MHC-bound peptides on antigen-presenting cells (APCs) by the T cell receptor (TCR). Activation of T lymphocytes is tightly regulated to acquire an appropriate immune response, as impaired T cell stimulation prevents the clearance of infectious pathogens (Zhang and Bevan, 2011). By contrast, persistent TCR triggering leads to the development of a unique state of T cells, known as exhaustion (Wherry, 2011). T cell exhaustion is found in chronic viral infections and tumors, in which T lymphocytes show compromised effector functions as indicated by impaired cytokine production, high expression of inhibitory receptors, and reduced cytotoxic activity (Wherry, 2011).

The TCR is a multiprotein complex that is exclusively expressed on the surface of T lymphocytes (Hedrick et al., 1984; Yanagi et al., 1984). Upon antigen recognition, Src-family kinases, such as lymphocyte-specific protein tyrosine kinase (Lck), are activated and proceed to phosphorylate immunoreceptor tyrosine-based activation motifs (ITAMs) on the TCR-associated CD3 molecules. The phosphorylation of CD3 molecules, especially CD3ζ, creates docking sites for zeta-chain-associated protein kinase (ZAP) 70. Engagement of the tandem SH2-domain of ZAP70 by phosphorylated ITAMs therefore enables ZAP70 to activate and phosphorylate the key mediators of TCR signaling, such as Linker for Activation of T cells (LAT), which serves as a nucleation center for downstream signaling molecules.

The engagement of the TCR takes place at the conjunction between a T cell and an APC, known as the immunological synapse (IS). The IS is characterized by the segregation of membrane receptors and intracellular molecules into three ring-like structures: central supramolecular activation cluster (cSMAC) composed of TCR and protein kinase C (PKC) θ, peripheral SMAC (pSMAC) formed by lymphocyte function-associated antigen (LFA) 1, and distal SMAC (dSMAC), rich in actin and CD45 (Grakoui et al., 1999; Monks et al., 1998)). Upon TCR engagement, signaling events are initially generated and propagated in TCR microclusters in the periphery of the synapse. Subsequently, the TCR microclusters are translocated to the cSMAC for termination of signaling, potentially via multivesicular body (MVB)-mediated lysosomal degradation of TCRs (Vardhana et al., 2010; Varma et al., 2006).

Protein-lipid interactions are important for the dynamics of the IS (Gagnon et al., 2012; Le Floc'h et al., 2013). Several studies indicate that anionic lipids, especially phosphatidylserine (PS), are involved in the binding of the cytoplasmic domain of CD3ε and CD3ζ to the cell membrane (Xu et al., 2008; Zhang et al., 2011), which in turn regulates their function. Likewise, many TCR downstream molecules, such as PKCθ and AKT (Huang et al., 2011; Melowic et al., 2007), rely on lipid binding for their full activation, highlighting the possibility that altering lipid distribution affects T cell activation. Interestingly, antigen stimulation has been shown to trigger local changes of PS in TCR microclusters (Gagnon et al., 2012). However, the functional consequences of active lipid regulation with regard to T cell activation are unknown.

Lipid distribution is regulated by three types of lipid translocases: flippase, which translocates lipids from the outer to the inner leaflet of the cell membrane; floppase, which is an outwardly directed translocase; and scramblase that is activated by Ca²⁺ and facilitates lipid transport across the membrane in a bidirectional fashion (Hankins et al., 2015). Flippase and floppase are mainly required for the ATP-dependent maintenance of asymmetric phospholipid distribution in membrane bilayers. With more than 90% of PS located in the inner leaflet of the membrane, it is unlikely that inactivation of these two lipid transporters induces rapid and robust redistribution of PS (Bevers and Williamson, 2010). Therefore, to study the active regulation of lipid redistribution, the work described herein focused on the well-defined lipid scramblase transmembrane protein (TMEM) 16F (Ehlen et al., 2013; Ousingsawat et al., 2015; Suzuki et al., 2010; Yang et al., 2012). TMEM16F, also called Anoctamin 6, was initially identified as a Ca²⁺-dependent scramblase by expression cloning and shown to mediate lipid transport across membranes (Suzuki et al., 2010). Of note, Scott syndrome, an inherited bleeding disorder due to defective phospholipid scramblase activity and lack of PS exposure on platelets (Weiss et al., 1979), was linked to loss-of-function mutations in the TMEM16F gene (Castoldi et al., 2011; Suzuki et al., 2010). Emerging evidence suggests that TMEM16F may have a dual function as Ca²⁺-dependent phospholipid scramblase and Ca²⁺-gated ion channel (Brunner et al., 2014; Malvezzi et al., 2013).

It is demonstrated herein that TMEM16F is the dominant scramblase in T cells and controls TCR-induced MVB formation. Moreover, it is demonstrated that TMEM16F deficiency leads to an accumulation of activated TCRs and associated signaling molecules due to impaired generation of MVBs. Remarkably, sustained TCR signaling causes severe exhaustion of T cells in chronic virus infection, which in turn leads to uncontrolled viral replication. These findings reveal a vital role of scramblase TMEM16F in termination of T cell activation, highlighting the importance of active lipid regulation to develop balanced immune responses.

Results

TMEM16F is the Dominant Lipid Scramblase in T Cells

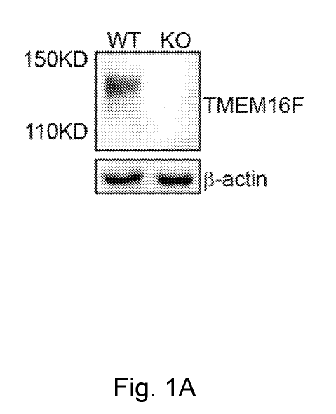

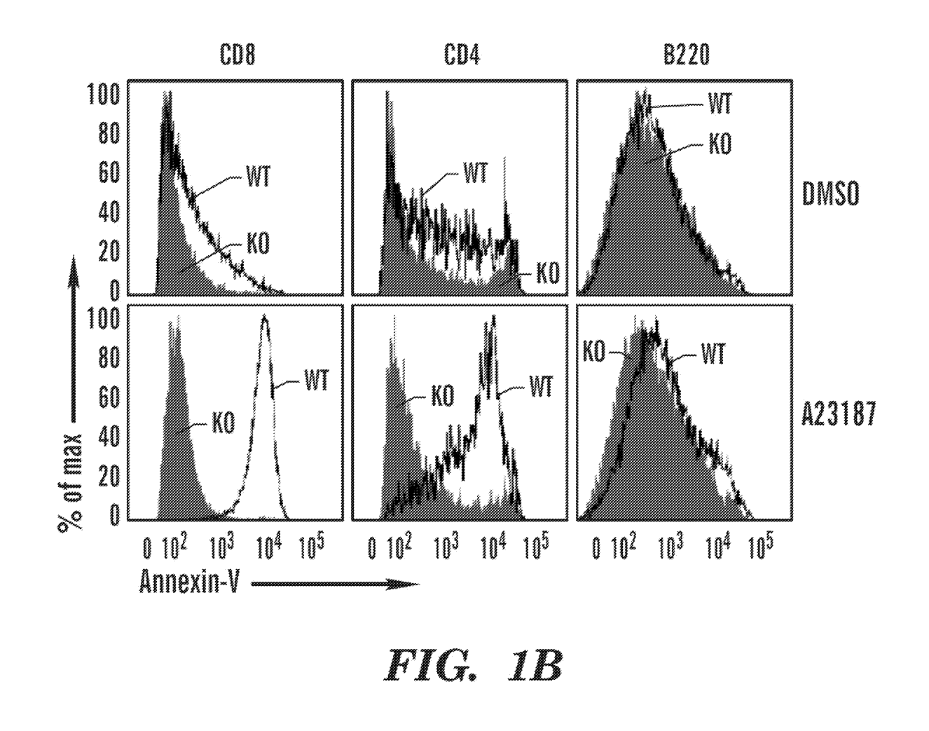

To test whether active regulation of lipid distribution controls the activation of T lymphocytes, the impact of scramblase on T cell stimulation was investigated. First, the expression of TMEM16F was determined in T cells. Immunoblot data showed that thymocytes from wild-type mice expressed TMEM16F, whereas it was absent in TMEM16F-deficient cells (FIG. 1A). To examine Ca²⁺-dependent scramblase activity, lymphocytes were incubated with a calcium ionophore (A23187) to induce exposure of PS on the cell surface. It was found that CD8 T cells lacking TMEM16F completely fail to scramble PS (FIG. 1B). TMEM16F was also required for scrambling in CD4 T cells, although a minor subpopulation seemed to be TMEM16F-independent (FIG. 1B). By contrast, TMEM16F did not facilitate PS exposure on B cells (FIG. 1B). Thus, TMEM16F is the dominant scramblase in T lymphocytes.

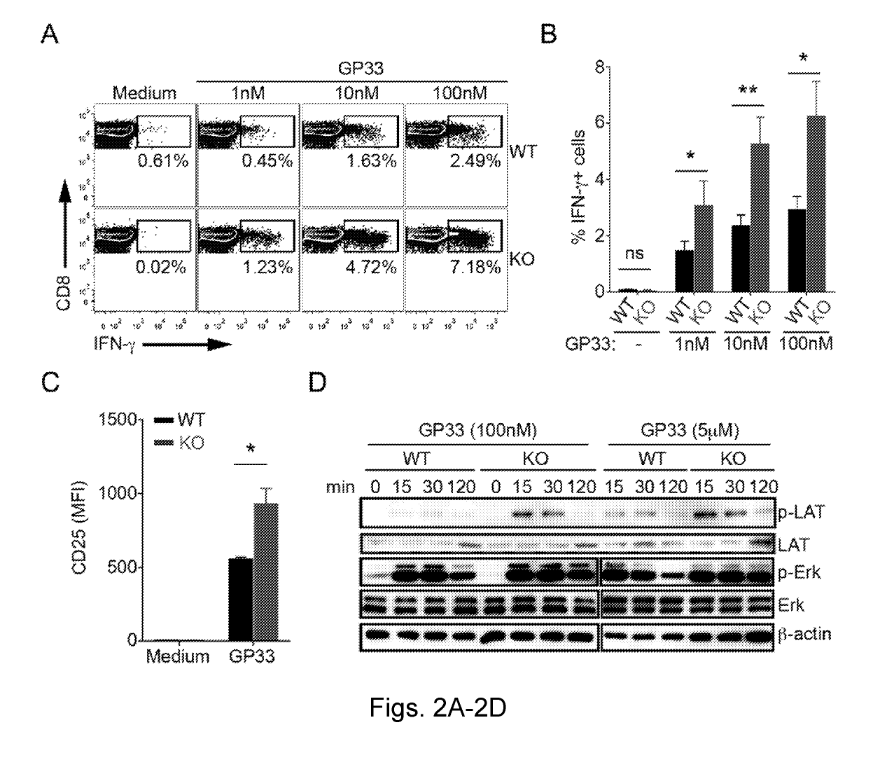

Lack of TMEM16F amplifies T cell activation. Next, the effect of TMEM16F deficiency on T cell activation was investigated. To generate an antigen-specific system, TMEM16F-KO mice were crossed with P14-transgenic animals that express a TCR specific for glycoprotein GP33 from lymphocytic choriomeningitis virus (LCMV). After stimulation of splenocytes with antigen, GP33 peptide, it was observed that IFN-γ production by naïve TMEM16F-deficient CD8 T cells was significantly increased (FIGS. 2A and 2B). Surface expression of the activation marker CD25 was similarly augmented on T cells lacking TMEM16F (FIG. 2C). In parallel, TCR downstream signaling was analyzed to gain in-depth resolution of T cell stimulation. TMEM16F-deficient T cells showed stronger and sustained phosphorylation of LAT (linker for activation of T cells) and ERK (extracellular signal regulated kinase), proximal and distal molecules in the TCR signaling cascade, respectively (FIG. 2D). Collectively, these data demonstrate that lack of TMEM16F causes increased T cell activation upon antigen stimulation.

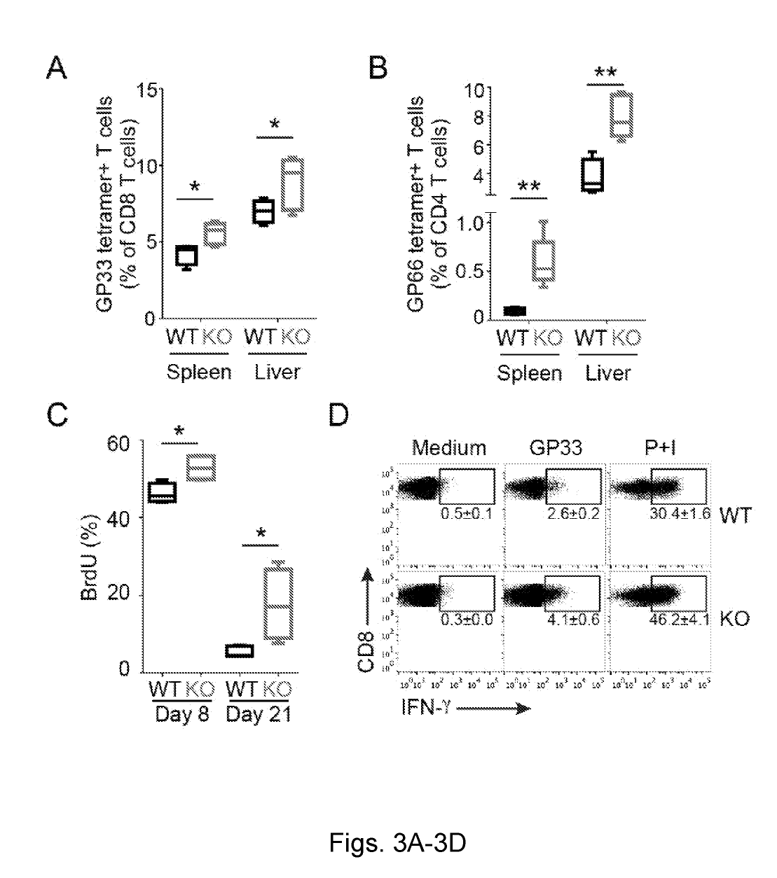

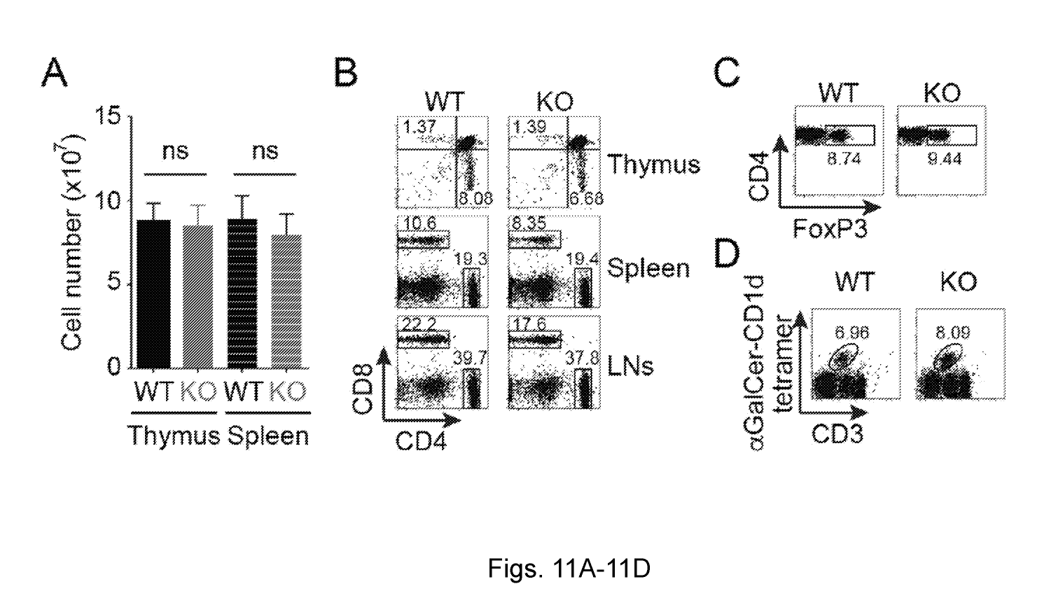

Increased T cell activation in TMEM16F-KO mice during early phase of chronic infection. Fine-tuning of T cell activation is crucial for effective immune responses. Persistent activation can lead to T cell exhaustion as seen in chronic viral infections (Wherry, 2011). Because sustained TCR signaling and over-activation of T cells was found in the absence of TMEM16F, the disease relevance of this mechanism, namely the immune response and protection against chronic viral infection in TMEM16F-deficient animals was next addressed. First, an effect of TMEM16F on T cell development was excluded. Numbers of CD8 and CD4 T cells in thymus and peripheral lymphatic organs, as well as frequencies of regulatory T cells (Tregs) and iNKT cells proved to be normal in TMEM16F-KO mice compared to wild-type littermate controls (FIGS. 11A-11D). Next, chronic infection was performed with LCMV clone 13 and increased frequencies of antigen-specific CD8 and CD4 T cells were found in TMEM16F-deficient mice within the first week after infection (FIGS. 3A and 3B). Increased activation of TMEM16F-deficient T cells was reflected by their augmented proliferation early after infection (FIG. 3C). While wild-type controls returned to background levels, T cells lacking TMEM16F remained highly proliferative at day 21 post infection (FIG. 3C). In addition, T cells in TMEM16F-KO animals produced higher amounts of IFN-γ (FIG. 3D). Taken together, these data demonstrate that restriction of TCR signaling by TMEM16F prevents excessive T cell responses during the early phase of chronic infection.

TMEM16F deficiency causes severe T cell exhaustion. Since persistent T cell stimulation can lead to dysfunctional T cell responses, T cell exhaustion was examined at later stages of chronic infection. Significantly increased expression of the exhaustion marker PD-1 was found on CD8 T cells from TMEM16F-deficient animals (FIGS. 4A and 4B). After stimulation with different CD8 T cell antigenic epitopes, co-production of IFN-γ and TNF-α was strikingly decreased when TMEM16F was lacking (FIGS. 4C and 4D). Similarly, antigen-specific CD4 T cells expressed higher amounts of PD-1, and produced less cytokines (FIGS. 4E-4H). Thus, the data indicate that initial and sustained T cell over-activation causes severe T cell exhaustion in TMEM16F deficiency.

In chronic LCMV infection, anti-viral T cell responses are controlled and balanced by two T cell populations: T-bet^{hi} and Eomes^{hi} cells (Kao et al., 2011; Paley et al., 2012; Staron et al., 2014). T-bet^{hi} cells represent a pool of precursors, and Eomes^{hi} cells are a terminally differentiated population. As persistent antigenic stimulation is linked to the transition of T-bet^{hi} to Eomes^{hi} cells (Kao et al., 2011; Paley et al.,2012), TMEM16F deficiency may break the balance between those two populations to eventually deplete T-bet^{hi} cells. As anticipated, later in chronic infection, T-bet expression was diminished while Eomes was complementarily increased in TMEM16F-deficient cells, indicating terminal differentiation of T cells (FIGS. 4I and 4J).

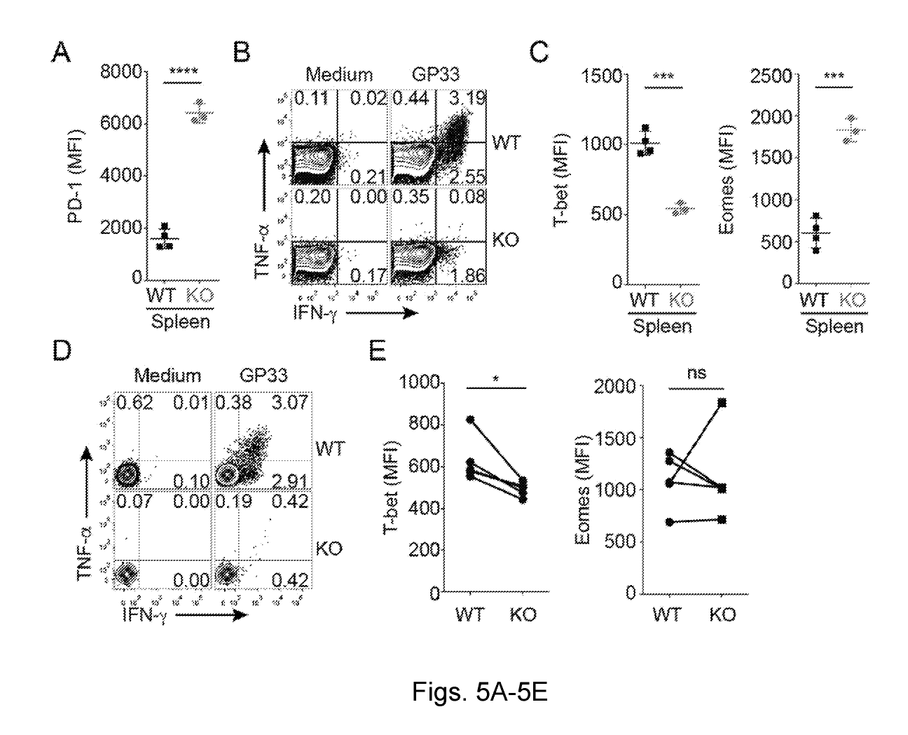

To confirm that TMEM16F acted directly in CD8 T cells, bone marrow chimera were generated using Rag1^{-/-} mice as recipients. Consistently, during chronic infection, TMEM16F-deficient CD8 T cells were severely exhausted (FIGS. 5A-5C). This defect proved to be T cell-intrinsic, as TMEM16F-deficient CD8 T cells exclusively failed to produce cytokines compared to WT in chronic infection of mixed BM chimera (FIG. 5D). Moreover, the T-bet^{hi} precursor pool was largely depleted when TMEM16F was lacking (FIG. 5E). Taken together, these results indicate that TMEM16F is vital to protect T lymphocytes from severe exhaustion during chronic infection.

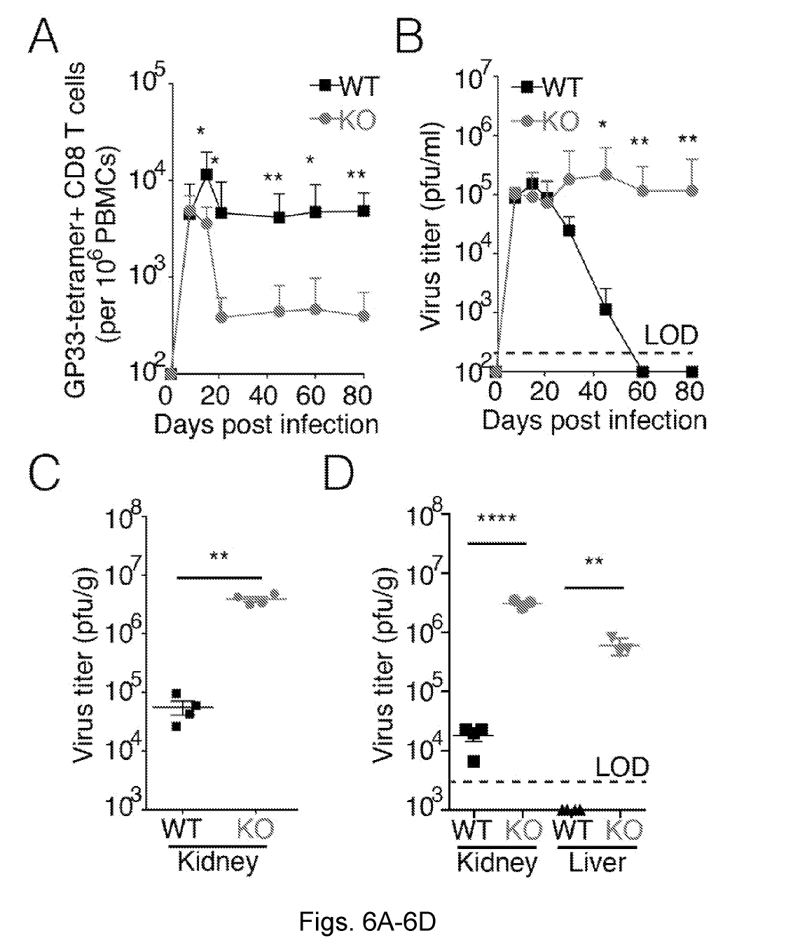

TMEM16F is required for control of chronic virus infection. Taking into account that the balance of T-bet^{hi} and Eomes^{hi} cell populations is vital to maintain the cytotoxic T lymphocyte (CTL) pool (Paley et al., 2012), a reduced number of antigen-specific CD8 T cells were observed in TMEM16F-KO mice (FIG. 6A). Since it is well established that CD8 T cells are essential to control LCMV clone 13 infection, the impaired maintenance of the CTL pool in TMEM16F-KO mice suggests an insufficiency to resolve the infection. In WT mice, virus in blood and tissues, such as the liver, was cleared two months after infection (FIG. 6B). However, TMEM16F deficiency leads to uncontrolled viremia (FIG. 6B), as well as failure of virus clearance in tissues (FIG. 6C and 6D). These results indicate that TMEM16F is critical to maintain a partial but effective anti-viral T cell response to limit chronic virus infection.

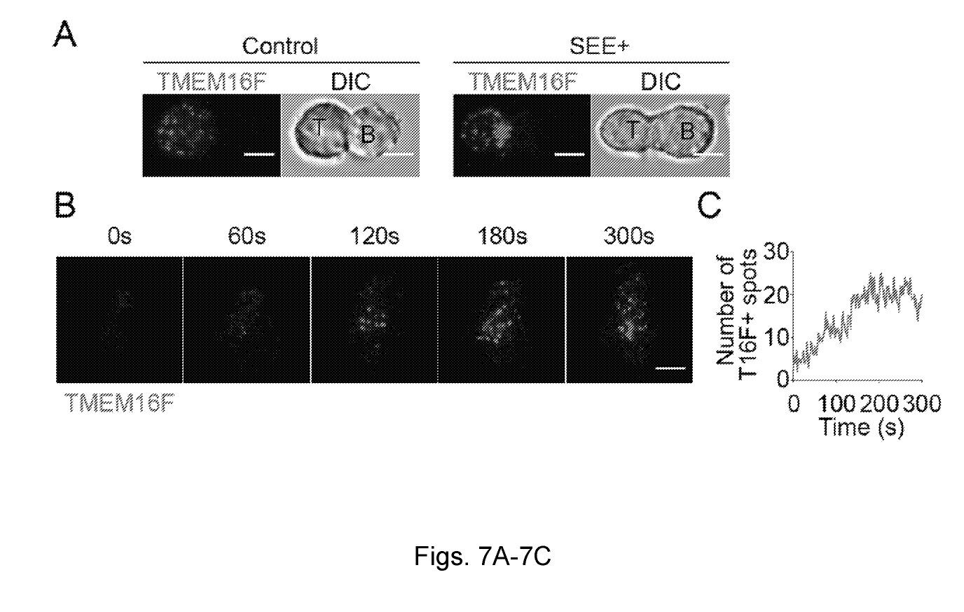

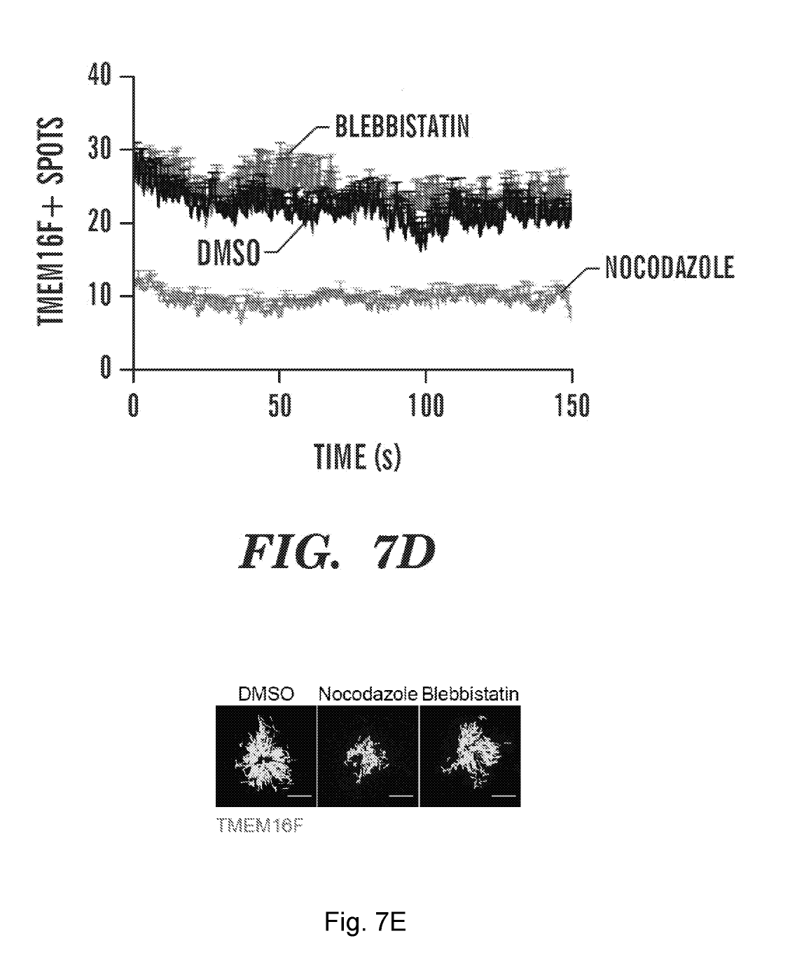

TMEM16F is recruited to the immunological synapse and resides in late endosomes. To elucidate the molecular mechanism how TMEM16F controls T cell activation, its cellular localization and function were tracked. To this end, T cells transduced with fluorescent TMEM16F were cocultured with antigen-presenting B cells. After antigen stimulation, TMEM16F was recruited to the IS as revealed by confocal microscopy (FIG. 7A). Of note, TMEM16F appeared mainly in intracellular structures instead of the plasma membrane (FIG. 7A). To kinetically demonstrate the recruitment of TMEM16F to the TCR activation site, TIRF (total internal reflection fluorescence) microscopy was used to visualize membrane proximal events of T cells on stimulatory coverslips (FIG. 7B and 7C). TMEM16F was found to be highly motile with a velocity reaching up to 1.5 μm/s, a speed similar to vesicle trafficking (data not shown). In order to test the mechanics of TMEM16F-positive vesicle movement, TIRF live imaging was performed while modulating the functions of the cytoskeleton. Inhibition of actin-myosin contractions by blebbistatin did not alter recruitment of TMEM16F to the T cell activation site (FIG. 7D and 7E). By contrast, treatment with nocodazole that disrupts the microtubule network reduced migration of TMEM16F vesicles, indicating the movement of TMEM16F was largely mediated by microtubules but not actin (FIGS. 7D and 7E).

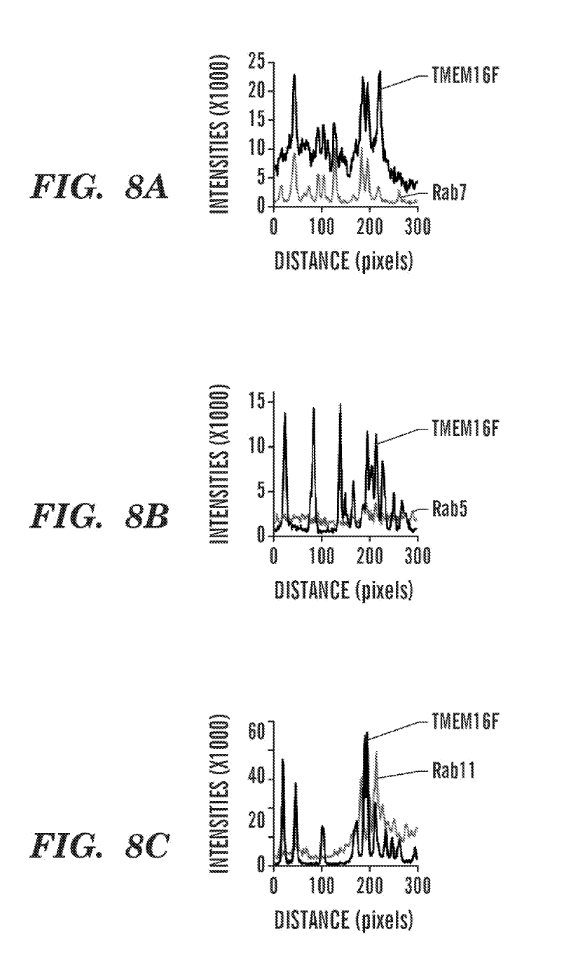

Therefore, in order to precisely define the subcellular compartment of TMEM16F, its colocalization was assessed with endosomal reporters in T cells, using image series taken from TIRF microscopy. Accordingly, Rab7 largely colocalized with TMEM16F, indicating that it mainly resides in late endosomes (FIG. 8A and data not shown). As controls, TMEM16F did not colocalize with the early endosomal marker Rab5 (FIG. 8B and data not shown), nor Rab11 that labels recycling endosomes (FIG. 8C and data not shown). Moreover, the localization of TMEM16F in a Rab7-positive compartment was independent of T cell activation status (data not shown). However, the abundance of TMEM16F in late endosomes increased upon TCR triggering (data not shown). In all, these findings reveal an unexpected localization of TMEM16F in late endosomes, pointing to the existence of an unidentified endosomal mechanism for TCR signal termination.

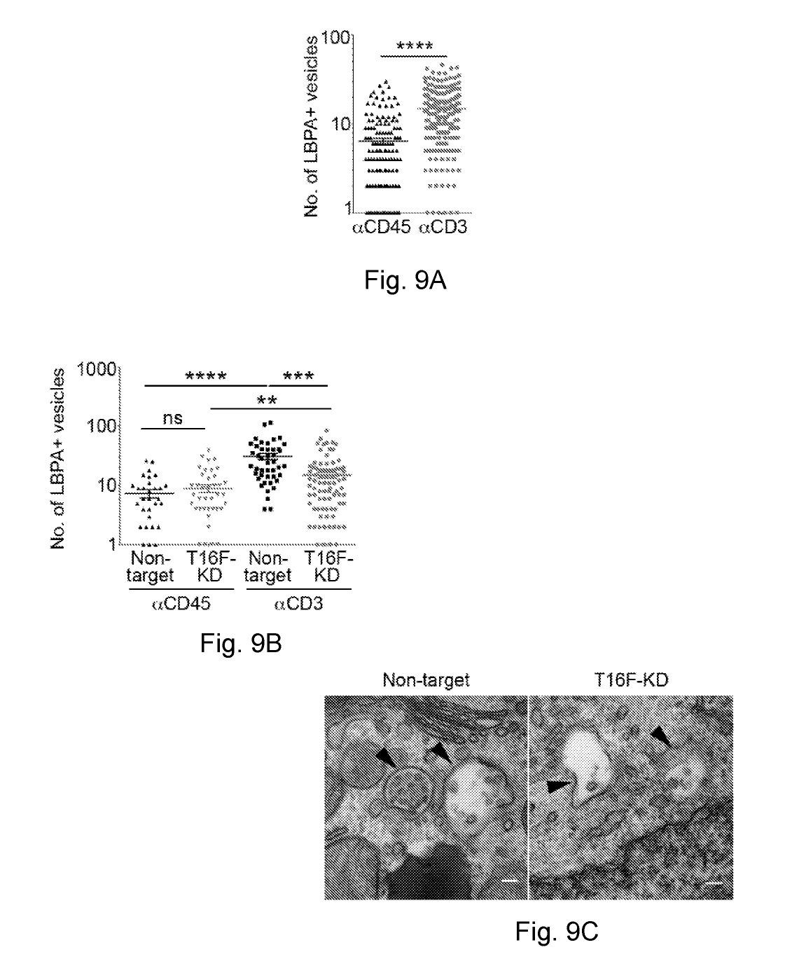

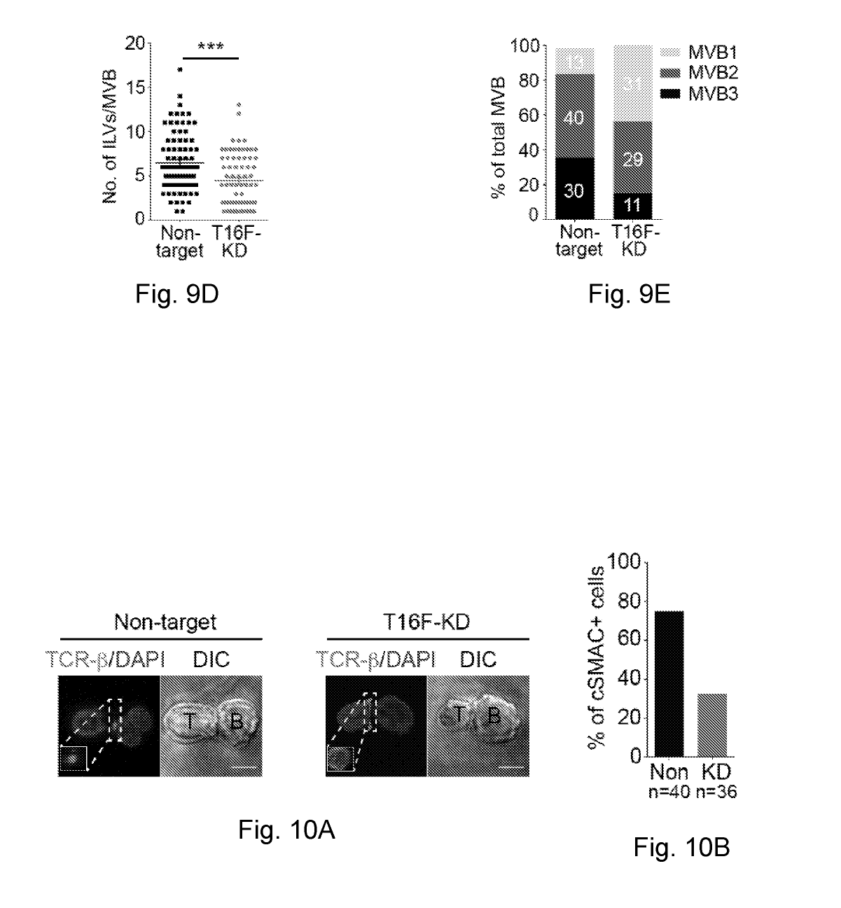

TMEM16F is involved in MVB formation upon TCR engagement. Late endosomes are also known as multivesicular bodies (MVBs), which are unique compartments that contain intraluminal vesicles (ILVs). The major role of MVBs is to degrade its contents via fusion with lysosomes. In T cells, MVBs have been associated with TCR degradation and signal termination (Vardhana et al., 2010; Varma et al., 2006). To address the impact of TMEM16F on MVB formation, stainings were performed for lysobisphosphatidic acid (LBPA), a marker for ILVs (Kobayashi et al., 1998). Interestingly, stimulation through the TCR greatly increased the number of LBPA-positive vesicles in T cells (FIG. 9B), indicating that TCR signaling enhances MVB formation. To evaluate whether TMEM16F is involved in this process, knockdown of TMEM16F was performed in T cells using shRNA. Lack of TMEM16F had no impact on the amount of LBPA-containing vesicles in unstimulated T cells, however, after TCR stimulation, the increase in LBPA-positive vesicles in controls was abrogated in TMEM16F-silenced T cells (FIG. 9B). Since the occurrence of ILVs in endosomes defines MVBs, the ultrastructure of the endosomal compartment in T cells lacking TMEM16F was investigated. Analysis by electron microscopy revealed a significantly reduced number of ILVs per MVB in TMEM16F-deficient T cells (FIGS. 9C and 9D). Following a classification of different MVB maturation stages (Vogel et al., 2015), MVB1, characterized by electron-translucent lumen and ≤3 ILVs, was strikingly more abundant in the absence of TMEM16F (FIG. 9E). Consequently, MVB2 (translucent lumen, >3 ILVs) and especially mature MVB3 (stained vacuole, >3 ILVs) were less numerous in TMEM16F-deficient T cells (FIG. 9E), showing that a defect in TMEM16F delays the progression of MVB development. Taken together, these data demonstrate that TMEM16F is involved in the increased generation of MVBs upon T cell activation.

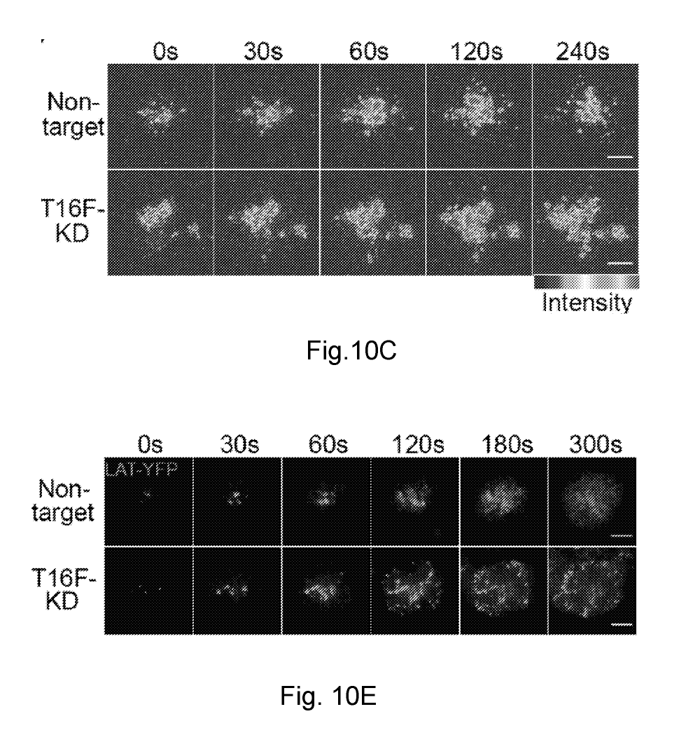

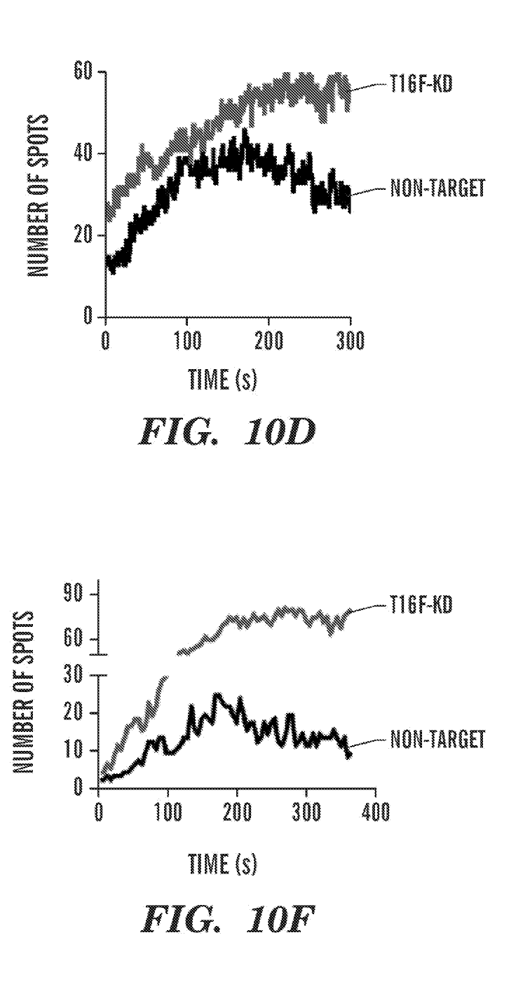

Impaired cSMAC formation and sustained TCR signaling in TMEM16F-deficient T cells. Since functional MVBs are involved in the development of the IS center where TCRs accumulate for signal termination (Vardhana et al., 2010; Varma et al., 2006), the impact of TMEM16F on cSMAC formation was investigated. Confocal microscopy followed b 3D reconstruction of the contact site between antigen-presenting B cells and responding T cells was performed to visualize the IS en face (FIGS. 10A and 10B). In control cells, the cSMAC formed as a dense spot of TCRs. By contrast, in TMEM16F-deficient T cells, TCRs remained in the periphery and failed to translocate to the IS centre (FIG. 10A). A defect in cSMAC formation has been shown to cause chronic TCR signaling (Vardhana et al., 2010). Thus, to functionally detect active TCR signaling, T cells were tranduced with the SH2 domain of ZAP70 that specifically binds to phosphorylated tyrosine residues in the CD3 signaling units of TCRs (Yudushkin and Vale, 2010). Using real-time TIRF microscopy, we demonstrate that active TCR signaling is sustained in TMEM16F-deficient T cells (FIGS. 10C and 10D). To further evaluate key events in TCR signal transduction the dynamics of LAT microclusters at the TCR activation site were examined. Notably, dissolution of these microclusters indicates signal termination. In accordance with the findings of increased LAT phosphorylation in T cells lacking TMEM16F (FIG. 2D), live imaging revealed an increase of newly formed LAT microclusters in TMEM16F-deficient T cells (FIGS. 10E and 10F). Moreover, whereas LAT microclusters subsided in control cells minutes after T cell activation, they were stable over time in T cells deficient for TMEM16F (FIGS. 10E and 10F). Together, these results reveal that TMEM16F is important to terminate TCR signaling by promoting cSMAC formation and subsequent degradation of active TCRs and associated signaling molecules.

Discussion

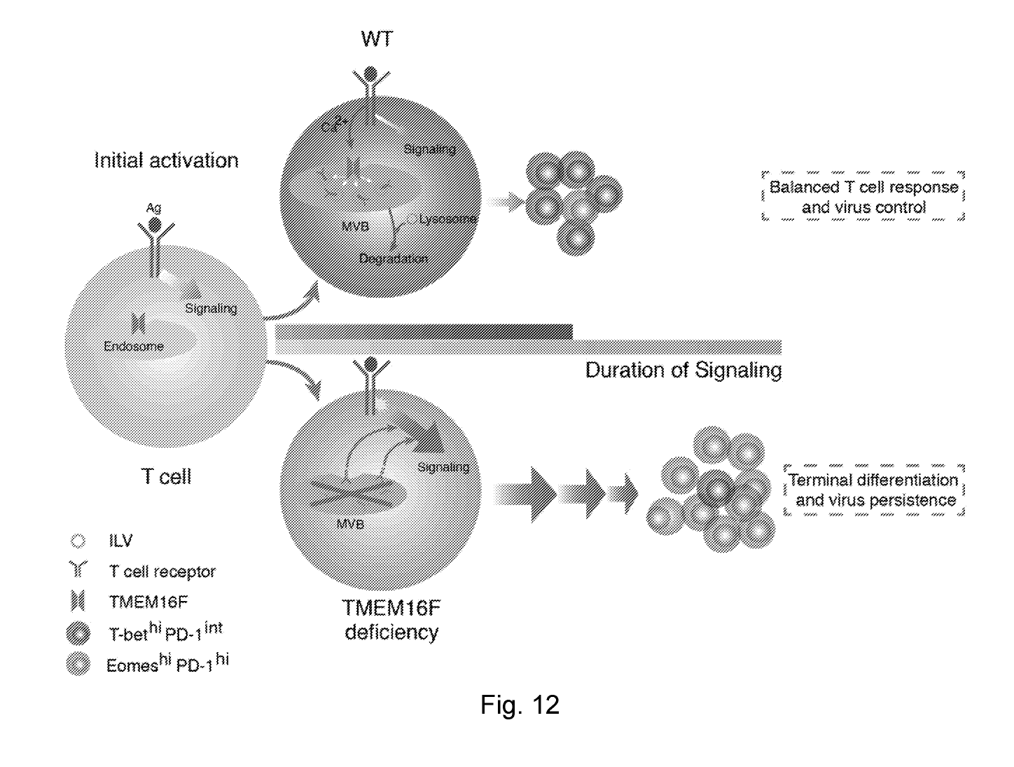

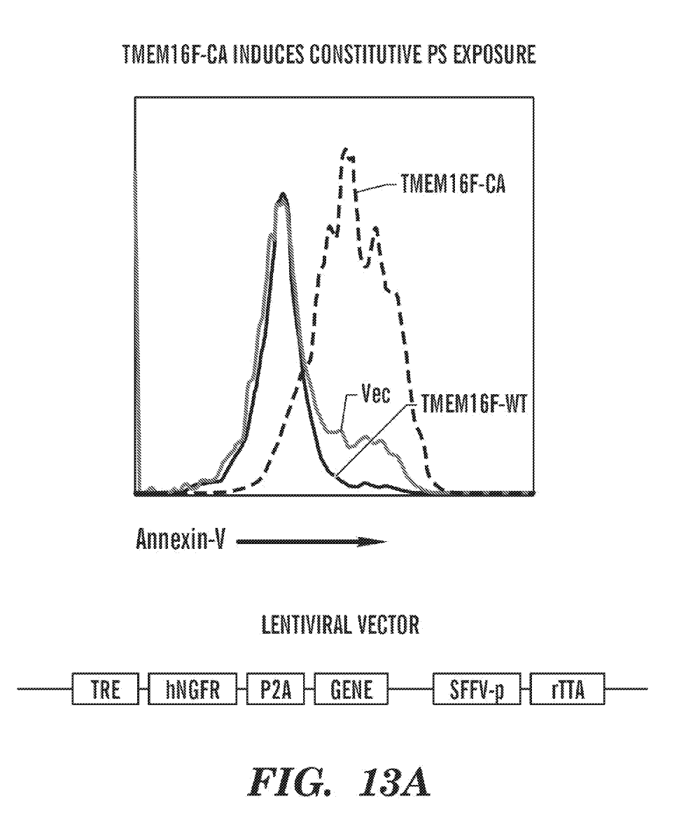

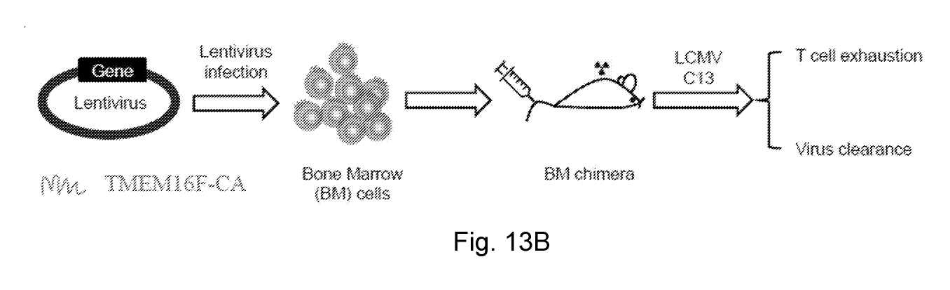

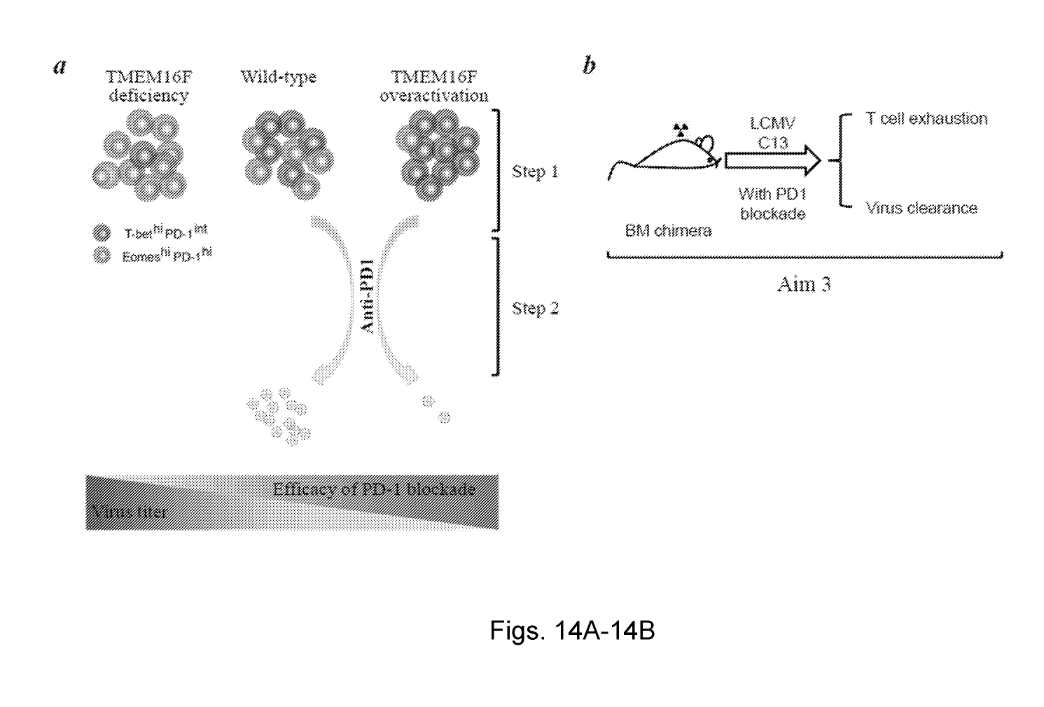

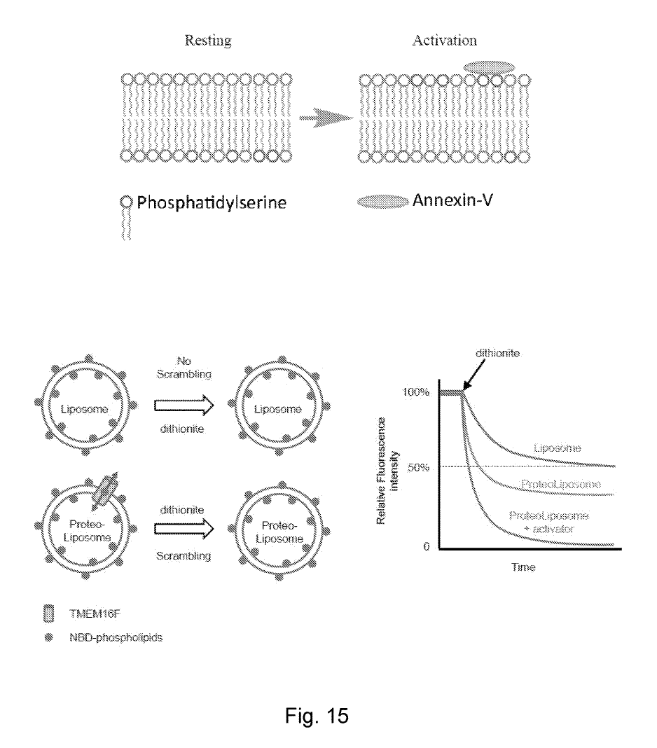

The data provided herein presents a novel mechanism to terminate TCR signaling by lipid scramblase TMEM16F. Without wishing to be bound by theory, it is contemplated herein that TCR engagement, via increased intracellular Ca²⁺ levels, activates scramblase TMEM16F in late endosomes to mediate the formation of MVBs. Newly generated MVBs sequester intracellular TCR signaling complexes for subsequent lysosomal degradation to terminate T cell activation. This TMEM16F-mediated checkpoint determines the duration of signaling and the proper ratio of T-bet^{hi} to Eomes^{hi} effector T cells to facilitate virus clearance. In the absence of TMEM16F, generation of MVBs is hampered, TCR signaling molecules accumulate, and T cell activation is sustained. Breaking the TMEM16F checkpoint leads to prolonged signaling that shifts the balance towards terminally differentiated Eomes^{hi} T cells and ultimate loss of virus protection (FIG. 12).

An unexpected localization of TMEM16F in endocytic compartments of T cells is described herein, providing mechanistic insights into endosomal lipid regulation and its functional consequences for cell signaling Accordingly, regulation of PS exposure was thought to be restricted to the plasma membrane. Based on the present findings, endosomes can be used as extra pool providing PS for accumulation on the outer leaflet of the plasma membrane. Indeed, previous studies have shown that calcium-mediated endosomal trafficking contributes to PS exposure upon apoptosis induction (Lee et al., 2013). However, since TMEM16F is dispensable for PS exposure during apoptosis (Segawa et al., 2014), the contributions of the endosomal pool to TMEM16F-dependent PS exposure remain to be evaluated.

Increasing evidence indicates that endosomes are not simply recipients of internalized receptors, but also sorting stations for either their degradation in lysosomes, or recycling to the plasma membrane, respectively (Irannejad et al., 2015). The balance between degradation and recycling dictates the outcome of signals initiated at the plasma membrane with regard to the quality of elicited responses, such as cell proliferation and survival. Furthermore, endosomes also serve as physical platform for signal transduction (Palfy et al., 2012). For example, endosomal epidermal growth factor receptors (EGFRs) contribute significantly to the overall EGFR signaling capacity (Fortian and Sorkin, 2014). Moreover, endosomes in T cells contain several key TCR signaling molecules (Benzing et al., 2013), whose activities are important for sustained TCR signaling and cell growth (Willinger et al., 2015; Yudushkin and Vale, 2010). Therefore, an important question is how endosomal signaling is terminated (Irannejad et al., 2015). One well-established mechanism is by sorting of activated receptor complexes into MVBs, mainly mediated by the endosomal sorting complex required for transport (ESCRT). In addition to ESCRT, collective evidence indicates that lipids also play key roles in MVB generation. For example, LBPA and ceramide induce membrane bending to promote formation of ILVs (Matsuo et al., 2004; Trajkovic et al., 2008). Interestingly, it is shown herein that the level of LBPA is increased upon TCR stimulation, which is correlated with the elevated number of MVBs in activated T cells that were identified by electron microscopy. Furthermore, the present work shows that lack of TMEM16F greatly reduces LBPA abundance only in activated T cells but not in resting cells, emphasizing an actively regulated process mediated by TMEM16F. A previous study using liposome assays showed that lipid composition affects the efficiency of ESCRT-mediated membrane scission during ILV generation (Wollert et al., 2009), indicating that ESCRT and locally enriched lipids might cooperatively control MVB formation. In addition, asymmetric distribution of lipids generates curvature during endosome trafficking and autophagosome formation (Hailey et al., 2010; Xu et al., 2013), a principle that can be applied to ILV generation as well. Without wishing to be bound by theory, it is contemplated herein that TMEM16F can locally regulate lipid redistribution in the endosomal membrane, which is able to modify the biophysical properties of the membrane for subsequent deformation (Stachowiak et al., 2013). In this context, it is shown herein that in the absence of TMEM16F, de novo generation of ILVs and MVBs is hampered, leading to accumulation of TCR signaling molecules and T cell activation that proceeds without restraint (FIG. 12).

Lastly, it is demonstrated herein that TMEM16F functions as an essential factor to restrict persistent infection with virus. It is known that continuous antigenic stimulation suppresses T cell activation in chronic viral infection (Staron et al., 2014). The present work reveals that TMEM16F plays a critical role in this process, as TMEM16F-deficient T cells are not able to curb proliferation in vivo. In addition, suppression of T cell responses during chronic viral infection was shown to protect the host from immunopathology (Ou et al., 2008). Surprisingly, overactivation of T cells in TMEM16F deficiency does not affect mortality, but instead results in severe T cell exhaustion and defective anti-viral responses, highlighting the sophisticated control of T cell responses in vivo. Significant advances have been made recently to better understand T cell exhaustion. It is commonly accepted that exhausted CD8 T cells still retain partial effector functions, which are crucial to restrain viral infection (Paley et al., 2012; Staron et al., 2014). In particular, the presence of T-bet^{hi} precursors in the exhausted population enables effective immunotherapy, owing to the fact that mainly T-bet^{hi} cells have the potential for reinvigoration after PD-1 pathway blockade (Blackburn et al., 2008). Remarkably, the presence of TMEM16F is a prerequisite for the maintenance of T-bet^{hi} cells during chronic infection, introducing TMEM16F as novel immune checkpoint to allow for a well-balanced immune response (FIG. 12).

Contemplated herein is targeting of TMEM16F to enhance its function in order to avoid exhaustion or reinvigorate T cell responses, e.g., thus permitting therapeutic strategies to improve efficacy of anti-PD-1 treatment against chronic diseases such as viral infection or cancer.

Materials and Methods

Mice. Generation of TME1116K^{-/-} mice has been reported previously (Ehlen et al., 2013). WT littermate mice were used as control. WT and TMEM16F^{-/-} mice were crossed to P14 TCR-transgenic animals. Rag1^{-/-} mice were purchased from the Jackson Laboratories (Bar Harbor, Me.). All animal procedures were approved by the Institutional Animal Care and Use Committee at Harvard Medical School.

Cells. MC57G and Jurkat cells (Clone E6-1) were obtained from ATCC. Raji and Jurkat cells were cultured in RPMI 1640 supplemented with 10% fetal bovine serum (FBS), L-glutamine (2 mM), β-mercaptoethanol (β-ME, 50 μM), penicillin/streptomycin (10 U/ml), sodium pyruvate (1 mM), and HEPES (100 mM). MC57G and 293T cells were cultured in Dulbecco's modified Eagle's medium supplemented with 10% FBS, L-glutamine (2 mM), penicillin and streptomycin (10 U/ml), and sodium pyruvate (1 mM). All cell culture reagents were obtained from Life Technologies unless otherwise noted.

Plasmids. TMEM16F-RFP fusion cDNA was subcloned into pHAGE2 lentiviral vector under control of the tetracycline responsive element (TRE) promoter. This vector harbors a human NGFR reporter separated by a P2A peptide fused in frame upstream of TMEM16F-RFP. PHR-mCherry-tSH2 (ZAP70), GFP-Rab5, GFP-Rab7, and GFP-Rab11 were obtained from Addgene. shRNA hairpin (TRCN0000134710) for human TMEM16F was obtained from the Sigma Mission library (Sigma-Aldrich). Non-target shRNA hairpin was used as a negative control (SHC002, Sigma-Aldrich). shRNA hairpins were subcloned into pLKO-Thy1.1 lentiviral vector using BamHI and NdeI.

PS Exposure. PS exposure assay was performed as previously reported (Suzuki et al., 2010). Briefly, cells were treated with 1 μM A23187 (Sigma-Aldrich) in Ca²⁺-free HBSS buffer (Life Technologies) at 37° C. for 15 min. After washing once with ice-cold HBSS buffer, cells were stained with FITC-annexin V (eBioscience) together with fluorochrome-conjugated antibodies on ice for 15 min. Cells were washed and analyzed by flow cytometry.

Flow Cytometry. Antibodies used for flow cytometry were purchased from BD Biosciences: CD8 (cat. no.: 553035), B220 (553088), CD44 (553133), CD3E (553066), CD25 (102012); Biolegend: CD4 (cat. no.: 100531), CD8 (100705), IFN-γ (505826), IL-2 (503808), T-bet (644808), Thy1.1 (202508), PD-1 (109109), CD45.1 (110722), human NGFR (345108); eBioscience: PD-1 (cat. no.: 50-2073-82), FoxP3 (12-5773-82), TNF-α (17-7321-81), Eomes (51-4875-82).

Viability dye (eBioscience) or propidium iodide (PI, Sigma-Aldrich) was used in all experiments to exclude dead cells. For surface antigens, cells were stained with antibody cocktail in Fluorescence-Activated Cell Sorting (FACS) buffer (PBS containing 0.5% BSA) on ice for 30 min. For intracellular cytokine staining, cells were stimulated with antigen, or PMA and Ionomycin (Sigma-Aldrich) for 3-5 h in the presence of GolgiStop™ (BD Biosciences), prior to surface antigen staining. Then, cells were fixed and stained for cytokines using Cytoflx/Cytoperm™ kit (BD Biosciences) according to manufacturer's protocol. Staining of FoxP3, T-bet, and Eomes was performed using Foxp3/Transcription Factor Staining Buffer Set (eBioscience) per manufacturer's instructions. Cells were processed using BD FACSCanto II™ (BD Biosciences). All flow cytometry data were analyzed with FlowJo™ (Treestar).

Immunoblot. Cells were lysed in loading buffer (62.5 mM Tris-HCl, 2% (w/v) SDS, 10% glycerol, and 0.01% β-ME (w/v)). For phosphorylation analysis, 1×10⁶ splenocytes from P14-WT or P14-TMEM16F^{-/-} mice were stimulated with GP33 for indicated time periods. Stimulation was terminated by adding 2× loading buffer (125 mM Tris-HCl, 4% (w/v) SDS, 20% glycerol, and 0.02% β-ME (w/v)). After boiling for 5 min, whole cell lysates were separated via SDS-PAGE, transferred to nitrocellulose membrane (Bio-Rad), stained with primary antibodies, followed by HRP-labeled anti-mouse IgG (cat. no.: sc-2031, Santa Cruz Biotechnology), or anti-rabbit IgG (ab6721, Abcam), and visualized with SuperSignal™ West Pico Chemiluminescent Substrate (Pierce). To detect TMEM16F expression, lysate from the same number of WT or TMEM16F^{-/-} thymocytes was used for loading. Antibodies used in the experiments were: phospho-LAT (07-278, Merck Millipore), LAT (sc-7948, Santa Cruz Biotechnology), phospho-Erk1/2 (4377, Cell Signaling Technology), Erk1/2 (C4695, Cell Signaling Technology), β-actin (ab8227, Abcam).

TIRF Microscopy. Sample preparation for TIRF microscopy was performed as previously reported with minor modifications (Bunnell et al., 2003). In brief, 0.01% poly-L-lysine-pretreated glass coverslips were coated with 10 μg/ml αCD45 (cat. no.: 304002, Biolegend), or αCD3 (317326, Biolegend) in PBS overnight at 4° C., or 2 h at 37° C. Jurkat cells were either transduced with TMEM16F-RFP lentivirus, or transfected with plasmids encoding indicated proteins by electroporation (Amaxa). Cells were then seeded on αCD45 or αCD3-coated coverslips containing imaging buffer (1.25 ml HEPES (1 M), 10 ml FBS, 38.75 ml RPMI without phenol red). When inhibitors were used, cells were pretreated with 1 μM nocodazole, or 1 μM blebbistatin (Sigma-Aldrich) for 30 min, and washed extensively before proceeding with TIRF. Unless otherwise indicated, images were taken every 2 s for 5 min. TIRF microscopy was done as previously reported (Cocucci et al., 2014).

Jurkat-Raji Conjugates and Confocal Microscopy. Raji cells were incubated for 2 h with 1 μg/ml of Staphylococcal enterotoxin E (SEE; Toxin Technology) at 37° C. After extensive washing, 1×10⁵ Raji cells were mixed with 1×10⁵ Jurkat cells and plated onto poly-L-lysine-coated coverslips in 24-well plates, incubated for 30 min at 37° C., and then fixed with 2% paraformaldehyde (PFA). To determine the localization of TMEM16F, reporter (TMEM16F-RFP) or TCR-β were used to distinguish Jurkat from Raji cells. For immunofluorescence assays, samples were permeabilized with 0.1% Triton X-100, blocked with 10% FBS plus 2% goat serum in PBS, and stained for TCR-Vβ8 (cat. no.: 555604, BD Biosciences), followed by goat anti-mouse IgG2b secondary antibody (A-21147, Life Technologies). All samples were mounted with ProLong® Gold Antifade Mountant with DAPI (Life Technologies). Images were taken using an Olympus FV1000™ confocal microscope. For 3D and z axis image reconstruction, 20 confocal sections, 0.4 μm apart, were assembled using ImageJ™/Fiji software (NIH).

Lentivirus Production, Titration, and Transduction. Lentiviral vectors were purified using GenElute™ HP Plasmid Kits (Sigma-Aldrich). For lentivirus production, 293T cells were seeded in 10 cm cell culture dishes. The following day, cells were transfected with a mixture of 45 μl TranslT®-293 Transfection Reagent (Minis), and 15 μg packaging plasmids. For shRNA, 7.5 μg pLKO-Thy1.1-shRNA hairpins, 6.75 μg PAX2, and 0.75 μg pCMV-VSVG were used for transfection. For TMEM16F-RFP, 12 μg pHAGE2 vector, 0.6 μg tat, 0.6 μg rev, 0.6 μg gag/pol, and 1.2 μg VSV-G were used for transfection. Lentivirus-containing supernatant was collected at 24, 48, and 72 h after transfection, passed through 0.45 μm filter (Millipore), and subsequently concentrated by ultracentrifugation at 17,000 rpm for 90 min. The virus pellet was dissolved in PBS.

Lentivirus stock was titrated on Jurkat cells using Thy1.1 (for shRNA) or human NGFR (for TMEM16F-RFP) as a reporter. Briefly, 1.5×10⁴ Jurkat cells were cocultured with serial dilutions of virus stock for 48 h in round-bottom 96-well plates. Infection rate was determined by flow cytometry analysis for reporter expression. The concentration of lentivirus was calculated using the following formula: virus/ml=percent infection (90%=0.9)×cell number (15000)×1000/X μl (where X is volume of virus added). The titer of lentivirus stocks was on average 10⁷-10⁸ infection units/ml.

For transduction, target cells were infected with multiplicity of infection (MOI) 1 in the presence of 8 μg/ml polybrene (Sigma-Aldrich). To induce TMEM16F-RFP expression, 1 μg/ml doxycycline (Sigma-Aldrich) was added to culture medium one day before analysis. Transduction efficiency for lentivirus was >98% for all experiments.

RNA Extraction and Real-Time PCR

Total RNA was extracted with TRIzol (Life Technologies) according to the manufacturer's instructions. High Capacity RNA-to-cDNA Kit (Applied Biosystems) was used for reverse transcription of purified RNA. All of the gene transcripts were quantified by real-time PCR with SYBR™ Green Master Mix (Bio-Rad) and a 7300 Real-Time PCR System (Applied Biosystems). The relative fold induction was calculated by the 2^{-ΔΔ}cycle threshold (CT) method. The sequences of primers for real-time PCR are:

Human TMEM16F, forward,
(SEQ ID NO: 1)
5′-GAAGAACAAGCCCGACCAGA-3′;
Human TMEM16F, reverse,
(SEQ ID NO: 2)
5′-CCACCTGGGTCACACTCTTC-3′;
Human GAPDH, forward,
(SEQ ID NO: 3)
5′-TGGGCTACACTGAGCACCAG-3′;
Human GAPDH, reverse,
(SEQ ID NO: 4)
5′-GGGTGTCGCTGTTGAAGTCA-3′.

LBPA Staining. LBPA staining was performed as previously reported with minor modifications (Varma et al., 2006). Briefly, Jurkat cells were stimulated on 10 μg/ml αCD45 or αCD3-coated coverslips for 5 min, and then fixed with 2% PFA for 30 min at RT. Cells were then blocked with 0.1% BSA for 30 min and stained with anti-LBPA antibody (cat. no.: Z-PLBPA, Echelon Biosciences) at 4° C. overnight in the presence of 0.05% saponin (Sigma-Aldrich), followed by Alexa 488-conjugated goat anti-mouse IgG1 secondary antibody (A-21121, Life Technologies). Samples were mounted in ProLong® Gold Antifade Mountant with DAPI.

Transmission Electron Microscopy. For T cell stimulation, 2×10⁶/ml Jurkat T cells were incubated with 10 μg/ml biotin-conjugated anti-human CD3E antibody (Biolegend), followed by cross-linking with 20 μg/ml streptavidin (Cell Signaling Technology) for 10 min at 37° C. Stimulation was stopped by adding the same volume of 2× fixative (2.5% glutaraldehyde, 1.25% paraformaldehyde, and 0.03% picric acid in 0.1 M sodium cacodylate buffer, pH 7.4). Cells were spun down at 1,500 rpm for 5 min. The cell pellet was fixed for 2 h at RT in the above fixative, washed in 0.1 M cacodylate buffer, and postfixed with 1% osmium tetroxide (OsO₄)/1.5% potassium ferrocyanide (KFeCN₆) for 1 h, washed in water and incubated in 1% aqueous uranyl acetate for 1 h. Cells were subsequently dehydrated in grades of alcohol (10 min each; 50%, 70%, 90%, 2×10 min 100%). The samples were then put in propylene oxide for 1 h and infiltrated overnight in a 1:1 mixture of propylene oxide and TAAB Epon (Marivac Canada Inc.; St. Laurent, Canada). The following day, the samples were embedded in TAAB Epon and polymerized at 60° C. for 48 h. Ultrathin sections (60 nm) were cut on a Reichert Ultracut-S™ microtome (Leica), transferred onto copper grids, stained with lead citrate, and examined in a JEOL 1200EX™ transmission electron microscope (JEOL), or a Tecnai G2 Spirit BioTWIN™ (FEI). Images were recorded with an AMT 2k™ CCD camera.

Image Processing. All images were processed using ImageJ™/Fiji software. For tracking of TMEM16F reaching the immunological synapse, TMEM16F-positive spots were examined using the Trackmate plug-in on Fiji™. The number of TMEM16F-positive spots was plotted using Prism 6.0™ Quantifications of LBPA-positive vesicles, LAT-GFP, and SH2-ZAP70-mCherry were done similarly. Intensity of SH2-ZAP70-mCherry was determined using intensity vs. time plug-in. For the colocalization of TMEM16F and Rab proteins, fluorescence intensity for each protein was measured on a line manually drawn along TMEM16F-positive vesicles, and values (pixel) were plotted. For Pearson's correlation analysis of the spatial relationship between TMEM16F and Rab proteins, six to seven movies (each has at least 300 frames) were used to determine the correlation coefficient (r) using the JACoP plug-in.

LCMV Infection and Virus Titer. Six to twelve week-old adult mice were intravenously infected with 4×10⁶ pfu of lymphocytic choriomeningitis virus (LCMV) clone 13. LCMV titer in serum and tissue homogenates was determined by focus assay. Briefly, MC57G cells were cultured in the presence of serially diluted samples for 48 h. Cell monolayers were fixed with 10% formalin (Sigma-Aldrich) in PBS, permeabilized by 1% Triton X-100, and then stained with anti-LCMV antibody VL-4 (cat. no.: BE0106-R005mg; Bio X Cell) and peroxidase-linked secondary antibody (Jackson ImmunoResearch). Plates were developed with SIGMA FAST OPD™ tablet (Sigma-Aldrich) at room temperature for 15-30 min. The reaction was terminated by washing the plates 5 times with distilled water.

Peptides and Tetramers. Peptides for LCMV antigens GP33 (KAVYNFATM) (SEQ ID NO: 5), GP276 (SGVENPGGYCL) (SEQ ID NO: 6), NP396 (FQPQNGQFI) (SEQ ID NO: 7), and GP61 (GLNGPDIYKGVYQFKSVEFD) (SEQ ID NO: 8) were purchased from AnaSpec (Fremont, Calif.). PE-conjugated H2D^{b} GP33-KAVYNFATM, and I-A^{b} LCMV GP66-77 tetramers were provided by the NIH Tetramer Facility (Atlanta, Ga.).

In vivo BrdU Incorporation. Mice were given 2 mg BrdU (Sigma-Aldrich) i.p. 20-24 h prior to tissue harvest and analysis. BrdU incorporation was evaluated by flow cytometry using the BrdU Flow Kit (BD Biosciences) following the manufacturer's instructions.

Bone Marrow Chimera. Bone marrow (BM) was prepared from tibias and femurs of WT (CD45.1) and TMEM/6F^{-/-} mice (CD45.2). Recipient Rag1^{-/-} mice were sublethally irradiated (450 rad) and reconstituted with either 3×10⁶ WT or TMEM/6F^{-/-}, or a 1:1 mixture of WT and TMEM16F^{-/-} BM cells. Recipient mice were treated with 2 mg/ml neomycin (Sigma-Aldrich) in drinking water for 4 weeks. Eight to twelve weeks after BM transfer, mice were infected with 4×10⁶ LCMV clone 13 i.v.

Statistical Analyses. All data are presented as means and standard errors of the mean (SEM). The statistical significance of differences was calculated by two-tailed Mann-Whitney test using Prism 6.0™ (GraphPad software). P-values≤0.05 were considered significant (*P<0.05; **P<0.01; ***P<0.001; ****P<0.0001)

### REFERENCES

Benzing, C., J. Rossy, and K. Gaus. 2013. Do signalling endosomes play a role in T cell activation? FEBS J 280:5164-5176.
Bevers, E. M., and P. L. Williamson. 2010. Phospholipid scramblase: an update. FEBS Lett. 584:2724-2730.
Blackburn, S. D., H. Shin, G. J. Freeman, and E. J. Wherry. 2008. Selective expansion of a subset of exhausted CD8 T cells by alphaPD-L1 blockade. Proc. Natl. Acad. Sci. USA. 105:15016-15021.
Brunner, J. D., N. K. Lim, S. Schenck, A. Duerst, and R. Dutzler. 2014. X-ray structure of a calcium-activated TMEM16 lipid scramblase. Nature 516:207-212.
Bunnell, S. C., V. A. Barr, C. L. Fuller, and L. E. Samelson. 2003. High-resolution multicolor imaging of dynamic signaling complexes in T cells stimulated by planar substrates. Sci. STKE 2003:PL8.
Castoldi, E., P. W. Collins, P. L. Williamson, and E. M. Bevers. 2011. Compound heterozygosity for 2 novel TMEM16F mutations in a patient with Scott syndrome. Blood 117:4399-4400.
Cocucci, E., R. Gaudin, and T. Kirchhausen. 2014. Dynamin recruitment and membrane scission at the neck of a clathrin-coated pit. Mol. Biol. Cell 25:3595-3609.
Ehlen, H. W., M. Chinenkova, M. Moser, H. M. Munter, Y. Krause, S. Gross, B. Brachvogel, M. Wuelling, U. Kornak, and A. Vortkamp. 2013. Inactivation of anoctamin-6/Tmem16f, a regulator of phosphatidylserine scrambling in osteoblasts, leads to decreased mineral deposition in skeletal tissues. J. Bone Miner. Res. 28:246-259.
Fortian, A., and A. Sorkin. 2014. Live-cell fluorescence imaging reveals high stoichiometry Grb2 binding to the EGF receptor sustained during endocytosis. J. Cell Sci. 127:432-444.
Gagnon, E., D. A. Schubert, S. Gordo, H. H. Chu, and K. W. Wucherpfennig. 2012. Local changes in lipid environment of TCR microclusters regulate membrane binding by the CD3epsilon cytoplasmic domain. J. Exp. Med. 209:2423-2439.
Grakoui, A., S. K. Bromley, C. Sumen, M. M. Davis, A. S. Shaw, P. M. Allen, and M. L. Dustin. 1999. The immunological synapse: a molecular machine controlling T cell activation. Science 285:221-227.
Hailey, D. W., A. S. Rambold, P. Satpute-Krishnan, K. Mitra, R. Sougrat, P. K. Kim, and J. Lippincott-Schwartz. 2010. Mitochondria supply membranes for autophagosome biogenesis during starvation. Cell 141:656-667.
Hankins, H. M., R. D. Baldridge, P. Xu, and T. R. Graham. 2015. Role of flippases, scramblases and transfer proteins in phosphatidylserine subcellular distribution. Traffic 16:35-47.
Hedrick, S. M., D. I. Cohen, E. A. Nielsen, and M. M. Davis. 1984. Isolation of cDNA clones encoding T cell-specific membrane-associated proteins. Nature 308:149-153.
Huang, B. X., M. Akbar, K. Kevala, and H. Y. Kim. 2011. Phosphatidylserine is a critical modulator for Akt activation. J. Cell Biol. 192:979-992.
Irannejad, R., N. G. Tsvetanova, B. T. Lobingier, and M. von Zastrow. 2015. Effects of endocytosis on receptor-mediated signaling Curr. Opin. Cell Biol. 35:137-143.
Kao, C., K. J. Oestreich, M. A. Paley, A. Crawford, J. M. Angelosanto, M. A. Ali, A. M. Intlekofer, J. M. Boss, S. L. Reiner, A. S. Weinmann, and E. J. Wherry. 2011.
Transcription factor T-bet represses expression of the inhibitory receptor PD-1 and sustains virus-specific CD8+ T cell responses during chronic infection. Nat. Immunol. 12:663-671.
Kobayashi, T., E. Stang, K. S. Fang, P. de Moerloose, R. G. Parton, and J. Gruenberg. 1998. A lipid associated with the antiphospholipid syndrome regulates endosome structure and function. Nature 392:193-197.
Le Floc'h, A., Y. Tanaka, N.S. Bantilan, G. Voisinne, G. Altan-Bonnet, Y. Fukui, and M. Huse. 2013. Annular PIP3 accumulation controls actin architecture and modulates cytotoxicity at the immunological synapse. J. Exp. Med. 210:2721-2737.
Lee, S. H., X. W. Meng, K. S. Flatten, D. A. Loegering, and S. H. Kaufmann 2013. Phosphatidylserine exposure during apoptosis reflects bidirectional trafficking between plasma membrane and cytoplasm. Cell Death Differ. 20:64-76.
Malvezzi, M., M. Chalat, R. Janjusevic, A. Picollo, H. Terashima, A. K. Menon, and A. Accardi. Ca2+-dependent phospholipid scrambling by a reconstituted TMEM16 ion channel Nat Commun 4:2367. Matsuo, H., J. Chevallier, N. Mayran, I. Le Blanc, C. Ferguson, J. Faure, N. S. Blanc, S. Matile, J. Dubochet, R. Sadoul, R. G. Parton, F. Vilbois, and J. Gruenberg. 2004. Role of LBPA and Alix in multivesicular liposome formation and endosome organization. Science 303:531-534.
Melowic, H. R., R. V. Stahelin, N. R. Blatner, W. Tian, K. Hayashi, A. Altman, and W. Cho. 2007. Mechanism of diacylglycerol-induced membrane targeting and acitvation of protein kinase C theta. J. Biol. Chem. 282:21467-21476.
Monks, C. R., B. A. Freiberg, H. Kupfer, N. Sciaky, and A. Kupfer. 1998. Three-dimensional segregation of supramolecular activation clusters in T cells. Nature 395:82-86.
Ou, R., M. Zhang, L. Huang, and D. Moskophidis. 2008. Control of virus-specific CD8+ T-cell exhaustion and immune-mediated pathology by E3 ubiquitin ligase Cbl-b during chronic viral infection. J. Virol. 82:3353-3368.
Ousingsawat, J., P. Wanitchakool, A. Kmit, A. M. Romao, W. Jantarajit, R. Schreiber, and K. Kunzelmann. 2015. Anoctamin 6 mediates effects essential for innate immunity downstream of P2X7 receptors in macrophages. Nat Commun 6:6245.
Paley, M. A., D. C. Kroy, P. M. Odorizzi, J. B. Johnnidis, D. V. Dolfi, B. E. Barnett, E. K. Bikoff, E. J. Robertson, G. M. Lauer, S. L. Reiner, and E. J. Wherry. 2012. Progenitor and terminal subsets of CD8+ T cells cooperate to contain chronic viral infection. Science 338:1220-1225.
Palfy, M., A. Remenyi, and T. Korcsmaros. 2012. Endosomal crosstalk: meeting points for signaling pathways. Trends Cell Biol. 22:447-456.
Segawa, K., S. Kurata, Y. Yanagihashi, T. R. Brummelkamp, F. Matsuda, and S. Nagata. 2014. Caspase-mediated cleavage of phospholipid flippase for apoptotic phosphatidylserine exposure. Science 344:1164-1168.
Smith-Garvin, J. E., G. A. Koretzky, and M. S. Jordan. 2009. T cell activation. Annu. Rev. Immunol. 27:591-619.
Stachowiak, J. C., F. M. Brodsky, and E. A. Miller. 2013. A cost-benefit analysis of the physical mechanisms of membrane curvature. Nat. Cell Biol. 15:1019-1027.
Staron, M. M., S. M. Gray, H. D. Marshall, I. A. Parish, J. H. Chen, C. J. Perry, G. Cui, M. O. Li, and S. M. Kaech. 2014. The Transcription Factor FoxO1 Sustains Expression of the Inhibitory Receptor PD-1 and Survival of Antiviral CD8 T Cells during Chronic Infection. Immunity 41:802-814.
Suzuki, J., M. Umeda, P. J. Sims, and S. Nagata. 2010. Calcium-dependent phospholipid scrambling by TMEM16F. Nature 468:834-838.
Trajkovic, K., C. Hsu, S. Chiantia, L. Rajendran, D. Wenzel, F. Wieland, P. Schwille, B. Brugger, and M. Simons. 2008. Ceramide triggers budding of exosome vesicles into multivesicular endosomes. Science 319:1244-1247.
Vardhana, S., K. Choudhuri, R. Varma, and M. L. Dustin. 2010. Essential role of ubiquitin and TSG101 protein in formation and function of the central supramolecular activation cluster. Immunity 32:531-540.
Varma, R., G. Campi, T. Yokosuka, T. Saito, and M. L. Dustin. 2006. T cell receptor-proximal signals are sustained in peripheral microclusters and terminated in the central supramolecular activation cluster. Immunity 25:117-127.
Vogel, G. F., H. L. Ebner, M. E. de Araujo, T. Schmiedinger, O. Eiter, H. Pircher, K. Gutleben, B. Witting, D. Teis, L. A. Huber, and M. W. Hess. 2015. Ultrastructural Morphometry Points to a New Role for LAMTOR2 in Regulating the Endo/Lysosomal System. Traffic 16:617-634.
Weiss, H. J., W. J. Vicic, B. A. Lages, and J. Rogers. 1979. Isolated deficiency of platelet procoagulant activity. Am. J. Med. 67:206-213.
Wherry, E. J. 2011. T cell exhaustion. Nat. Immunol. 12:492-499.
Willinger, T., M. Staron, S. M. Ferguson, P. De Camilli, and R. A. Flavell. 2015. Dynamin 2-dependent endocytosis sustains T-cell receptor signaling and drives metabolic reprogramming in T lymphocytes. Proc. Natl. Acad. Sci. USA. 112:4423-4428.
Wollert, T., C. Wunder, J. Lippincott-Schwartz, and J. H. Hurley. 2009. Membrane scission by the ESCRT-III complex. Nature 458:172-177.
Xu, C., E. Gagnon, M. E. Call, J. R. Schnell, C. D. Schwieters, C. V. Carman, J. J. Chou, and K. W. Wucherpfennig. 2008. Regulation of T cell receptor activation by dynamic membrane binding of the CD3epsilon cytoplasmic tyrosine-based motif. Cell 135:702-713.
Xu, P., R. D. Baldridge, R. J. Chi, C. G. Burd, and T. R. Graham. 2013. Phosphatidylserine flipping enhances membrane curvature and negative charge required for vesicular transport. J. Cell Biol. 202:875-886.
Yanagi, Y., Y. Yoshikai, K. Leggett, S. P. Clark, I. Aleksander, and T. W. Mak. 1984. A human T cell-specific cDNA clone encodes a protein having extensive homology to immunoglobulin chains. Nature 308:145-149.
Yang, H., A. Kim, T. David, D. Palmer, T. Jin, J. Tien, F. Huang, T. Cheng, S. R. Coughlin, Y. N. Jan, and L. Y. Jan. 2012. TMEM16F forms a Ca2+-activated cation channel required for lipid scrambling in platelets during blood coagulation. Cell 151:111-122.
Yudushkin, I. A., and R. D. Vale. 2010. Imaging T-cell receptor activation reveals accumulation of tyrosine-phosphorylated CD3zeta in the endosomal compartment. Proc. Natl. Acad. Sci. USA. 107:22128-22133.
Zhang, H., S. P. Cordoba, O. Dushek, and P. A. van der Merwe. 2011. Basic residues in the T-cell receptor zeta cytoplasmic domain mediate membrane association and modulate signaling Proc. Natl. Acad. Sci. USA. 108:19323-19328.
Zhang, N., and M. J. Bevan. 2011. CD8(+) T cells: foot soldiers of the immune system. Immunity 35:161-168.

### Example 2

T-cell exhaustion is the phenomenon that occurs when persistently activated T-cells (caused by continual interaction between T-cell receptor and cognate antigen) causes poor T effector cell function, and sometimes results in cell death. T-cell exhaustion has also been implicated in dysfunction memory T-cell establishment, and can result in epitope deletion. Overall, the picture emerges where T-cell exhaustion significantly compromises adaptive immunity, especially for chronic conditions such as cancer and viral infection.

Programmed Death-1 (PD-1) receptor is a regulator of T-cell exhaustion and survival; when PD-1 interacts with PD-L1/PD-L2 (where L means Ligand; PD-L1/2 molecules are expressed on the surfaces of a number of cell and tissue types), its downstream effectors help prevent proliferation, cell survival (inducement of apoptosis), and protein synthesis (including IL-2 production). PD-1, and therefore tissue tolerance and T-cell exhaustion, has become a potential therapeutic target for the treatment of cancer, viral infections, and autoimmune diseases.

The majority of therapeutic molecules (typically antibodies) being pursued by pharmaceutical companies antagonize the PD-1/PD-L1 or PD-L2 interaction for the treatment of cancer. Described herein is the characterization and modulation of a protein regulator upstream of PD-1, namely the phospholipid scramblase TMEM16F. It is described herein that knocking TMEM16F down or out leads to a significant 2-fold upregulation of PD-1 on activated T-cells. Additionally, TMEM16F knockout mouse-derived T-cells have increased expression of IFN-γ and increased T-cell activation, but also increased expression of several exhaustion markers (of which PD-1 is an example). Together, these effects combine to decrease the ability of the knockout mice to control chronic (21 days post inoculation) infection. These results indicate the synergy between PD-1 blockade and TMEM16F agonism, and it is specifically contemplated herein that TMEM16F be targeted to enhance its function to avoid exhaustion or reinvigorate T cell responses, thus permitting therapeutic strategies to improve efficacy of anti-PD-1 treatment against chronic disease such as viral infection or cancer.

1. A method of treating a viral infection or cancer in a subject in need thereof, the method comprising administering an agonist of TMEM16F to the subject.

2. The method of claim 1, whereby T cell exhaustion is inhibited.

3. (canceled)

4. (canceled)

5. (canceled)

6. The method of claim 1, wherein the viral infection is an HIV or hepatitis B virus (HBV).

7. The method of claim 1, wherein the cancer is selected from the group consisting of:
a recurrent cancer; melanoma; NSCLC; renal cell carcinoma; Hodgkin lymphoma; and bladder cancer.

8. The method of claim 7, wherein the agonist of TMEM16F is administered before or concurrently with another cancer treatment.

9. (canceled)

10. (canceled)

11. The method of claim 7, wherein the subject is further administered an immune checkpoint therapy.

12. The method of claim 11, wherein the immune checkpoint therapy is selected from the group consisting of:
an anti-PD-1 therapy; an anti-CTLA4 therapy; an anti-PD-L1 therapy; an anti-TIM-3 therapy; and an anti-LAG-3 therapy.

13. The method of claim 2, wherein
a. the anti-PD-1 therapy is selected from the group consisting of nivolumab; and pembrolizumab;
b. the anti-CTLA4 therapy is ipilimumab;
c. the anti-PD-L1 therapy is atezolizumab;
d. the anti-TIM3 therapy is TSR-022; or
e. the anti-LAG3 therapy is BMS-986016.

14. (canceled)

15. (canceled)

16. (canceled)

17. (canceled)

18. The method of claim 1, wherein the subject is further administered a CAR-T cell.

19. A method of administering a vaccine to a subject in need thereof, the method comprising administering:
the vaccine;
and an agonist of TMEM16F to the subject.

20. The method of claim 19, wherein the vaccine and the agonist of TMEM16F are administered sequentially.

21. The method of claim 19, wherein the vaccine and the agonist of TMEM16F are administered concurrently.

22. The method of claim 19, wherein the subject is a subject with reduced immune function.

23. The method of claim 22, wherein the subject with reduced immune function is a subject with a chronic disease.

24.-40. (canceled)

41. A method of treating an autoimmune or inflammatory disease in a subject in need thereof, the method comprising administering an inhibitor of TMEM16F to the subject.

42.-79. (canceled)

80. The method of claim 1, wherein the agonist of TMEM16F is a TMEM16F polypeptide, a nucleic acid encoding a TMEM16F polypeptide, or an SSRI inhibitor.

81. The method of claim 1, wherein the SSRI inhibitor is fluoxetine, sertraline, paroxetine, citalopram, escitalopram, fluvoxamine, dapoxetine, indalpine, zimelidine, cericlamine, or panuramine.
